(19) United States
(12) Patent Application Publication
(10) Pub. No.: US 2014/0113981 A1
(43) Pub. Date: Apr. 24, 2014

(54) Catalyst for the Conversion of Syngas to Olefins and Preparation Thereof

(71) Applicant: BASF SE, Ludwigshafen (DE)

(72) Inventors: Bilge Yilmaz, New York, NY (US); Ulrich Müller, Neustadt (DE); Xinhe Bao, Dalian (CN); Weiping Zhang, Dalian City (CN); Dirk de Vos, Holsbeek (BE); Takashi Tatsumi, Kawasaki (JP); Feng-Shou Xiao, Changchun (CN); Hermann Gies, Sprockhovel (DE); Hiroyuki Imai, Tokyo (JP)

(73) Assignee: BASF SE, Ludwigshafen (DE)

(21) Appl. No.: 14/056,002

(22) Filed: Oct. 17, 2013

Related U.S. Application Data
(63) Continuation of application No. PCT/CN2012/083209, filed on Oct. 19, 2012.

(51) Int. Cl. C07C 1/04; B01J 29/76

(52) U.S. Cl. CPC C07C 1/044; B01J 29/76
USPC 518/721; 502/74; 518/715

(57) ABSTRACT

Described is a process for the production of a pillared silicate. The process comprises (i) providing a layered silicate; (ii) interlayer expanding the layered silicate provided in step (i) comprising a step of treating the layered silicate with one or more swelling agents; (iii) treating the interlayer expanded silicate obtained in step (ii) with one or more hydrolyzable silicon containing compounds; (iv) treating the interlayer expanded compound obtained in step (iii) with an aqueous solution to obtain a pillared silicate; (v) removing at least a portion of the one or more swelling agents from the pillared silicate obtained in step (iv); and (vi) impregnating the pillared silicate obtained in step (v) with one or more elements selected from the group consisting of Fe, Ru, Ir, and combinations of two or more thereof. Also described is a pillared silicate optionally obtainable from said process and its use, in particular, in a process for the production of one or more olefins according to the invention.

### CROSS-REFERENCE TO RELATED APPLICATIONS

This application claims the benefit of priority under 35 U.S.C. §§120 and 365(c) to PCT Application No. PCT/CN2012/083209, filed Oct. 19, 2012, the entire content of which is incorporated herein by reference in its entirety.

### TECHNICAL FIELD

The present invention relates to a process for the production of a pillared silicate compound as well as to a pillared silicate compound obtainable from such a process. Furthermore, the present invention relates to a pillared silicate compound per se as well as to a process for the production of one or more olefins using the aforementioned pillared silicate compounds. Finally, the present invention also relates to the use of the aforementioned pillared silicate compounds.

### BACKGROUND

In the field of catalysis, as well as in the field of processes involving adsorption and/or absorption of chemical compounds, providing novel framework topologies with novel pore architectures plays a crucial role in the development of catalysts, catalyst components, and catalyst support materials displaying novel reactivity and/or improved performance. In this respect, besides efforts which have been invested into creating novel three-dimensional zeolitic frameworks from organo-template-mediated self-organization processes as may for example be achieved in hydrothermal synthetic procedures, efforts have been made to create micro- and/or macroporous structures starting from layered materials, and, in particular, layered silicates by chemical bonding of the layers with one another thus affording three-dimensional frameworks. In this respect, the condensation of layered silicates into new zeolitic frameworks via topotactic procedures may be mentioned. For achieving greater degrees of interlayer expansion, methods may also be mentioned wherein pillaring agents are introduced into layered materials. In this respect, it has been found that the use of auxiliary agents which are intercalated between the layers and later at least in part removed from the pillared materials may be employed for achieving greater degrees of interlayer expansion.

Thus, as regards the aforementioned methods, the synthesis and characterization of products resulting from the reaction of selected layered silicates having the MWW, FER, CDO, and MCM-47 topologies with diethoxydimethyl silane as disclosed in P. Wu et al., J. Am. Chem. Soc. 2008, vol. 130, pp. 8178-8187 may be mentioned.

EP 0 626 200 A1, on the other hand, relates to the production of silica-pillared micas which may be produced by intercalating an organosilicon oligomer/precursor into a layered fluoromica, followed by calcination thereof. WO 2010/100191 A1, on the other hand, discloses a process for the preparation of pillared silicates employing hydrothermal conditions. Additionally, O.-Y. Kwon et al., Bull. Korean Chem. Soc. 1999, vol. 20, pp. 69-75, concerns silica-pillared H-kenyaites produced by interlamellar base catalyzed-reaction of tetraethylorthosilicate wherein the intercalation of tetraethylorthosilicate is conducted with the aid of octylamine employed in a pre-swelling process.

Despite the results which have been achieved for obtaining interlayer expanded three-dimensional structures from such pillaring processes, there remains the need for novel structures and materials displaying unprecedented chemical properties and reactivities for use in various applications and in particular for use in the increasingly important field of catalysis and in particular of heterogeneous catalysis in which microporous structures such as those found in zeolitic materials have found a very large number of applications.

### SUMMARY

Aspects of a first embodiment are directed to a process for the production of a pillared silicate. In a first embodiment, the process comprises (i) providing a layered silicate; (ii) interlayer expanding the layered silicate provided in step (i) comprising a step of treating the layered silicate with one or more swelling agents; (iii) treating the interlayer expanded silicate obtained in step (ii) with one or more hydrolyzable silicon containing compounds; (iv) treating the interlayer expanded compound obtained in step (iii) with an aqueous solution to obtain a pillared silicate; (v) removing at least a portion of the one or more swelling agents from the pillared silicate obtained in step (iv); and (vi) impregnating the pillared silicate obtained in step (v) with one or more elements selected from the group consisting of Fe, Ru, Ir, and combinations of two or more thereof.

In a second embodiment, the process of the first embodiment is modified, wherein the process further comprisises (vii) drying the impregnated pillared silicate; and/or (viii) calcining the impregnated and optionally dried pillared silicate.

In a third embodiment, the process of the first and second embodiments is modified, wherein the process further comprises (ix) treating the impregnated and optionally dried and/or optionally calcined pillared silicate with a reducing agent.

In a fourth embodiment, the process of the first through third embodiments is modified, wherein the interlayer expanded silicate obtained in step (ii) is dehydrated prior to step (iii).

In a fifth embodiment, the process of the first through fourth embodiments is modified, wherein the layered silicate provided in step (i) comprises one or more layered silicate compounds selected from the group consisting of MCM-22, PREFER, Nu-6(2), CDS-1, PLS-1, MCM-47, ERS-12, MCM-65, RUB-15, RUB-18, RUB-20, RUB-36, RUB-38, RUB-39, RUB-40, RUB-42, RUB-51, BLS-1, BLS-3, ZSM-52, ZSM-55, kanemite, makatite, magadiite, kenyaite, revdite, montmorillonite, and combinations of two or more thereof.

In a sixth embodiment, the process of the first through fifth embodiments is modified, wherein the layered silicate provided in step (i) is isomorphously substituted.

In a seventh embodiment, the process the first through sixth embodiments is modified, wherein the one or more swelling agents used in step (ii) comprise one or more compounds selected from the group consisting of cationic surfactants.

In an eighth embodiment, the process of the first through seventh embodiments is modified, wherein the cationic surfactant contains 1 to 4 alkyl chains having 4 C-atoms or more.

In a ninth embodiment, the process of the first through eighth embodiments is modified, wherein the 1 to 4 alkyl chains having 4 C-atoms or more include one or more C₄-C₂₆ alkyl chains.

In a tenth embodiment, the process of the first through ninth embodiments is modified, wherein the cationic surfactant comprises one or more tetraalkylammonium compounds.

In an eleventh embodiment, the process of the first through tenth embodiments is modified, wherein the hydrolyzable silicon containing compound comprises one or more silicon compounds X¹X²SiX³X⁴, wherein X¹, X², X³, and X⁴ independently from one another represent a leaving group, wherein the leaving group X¹, X², X³, and X⁴ may be the same or different from one another.

In a twelfth embodiment, the process of the first through eleventh embodiments is modified, wherein in step (iii) the treatment is conducted at a temperature in the range of from 25° C. to the refluxing temperature of the hydrolizable silicon containing compound in the mixture provided in step (iii).

In a thirteenth embodiment, the process of the first through twelfth embodiments is modified, wherein in step (iii) the treatment is conducted for a period of from 1 to 72 h.

In a fourteenth embodiment, the process of the first through thirteenth embodiments is modified, wherein the aqueous solution used in step (iv) has a pH of from 5 to 10.

In a fifteenth embodiment, the process of the first through fourteenth embodiments is modified, wherein step (v) includes a calcination step for removing at least a portion of the swelling agent.

In a sixteenth embodiment, the process of the first through fifteenth embodiments is modified, wherein the calcination in step (viii) is conducted at a temperature ranging from 400 to 950° C.

In a seventeenth embodiment, the process of the first through sixteenth embodiments is modified, wherein the one or more sources for the one or more elements in step (vi) comprise one or more metal compounds selected from the group consisting of metal salts, metal complexes, organometallic compounds, and combinations of two or more thereof.

In an eighteenth embodiment, the process the first through seventeenth embodiments is modified, wherein the metal complexes comprise a ligand selected from the group consisting of mono-, bi-, tri-, tetra-, penta-, and hexadentate ligands, including combinations of two or more thereof.

In a nineteenth embodiment, the process of the first through eighteenth embodiments is modified, wherein step (vi) is repeated one or more times.

In a twentieth embodiment, the process of the first through nineteenth embodiments is modified, wherein the reducing agent in step (ix) comprises one or more compounds selected from the group consisting of hydrogen, hydrides, metals, sulfites, phosphites, hypophosphites, hydrazine, and combinations of two or more thereof.

In a twenty-first embodiment, the process of the first through twentieth embodiments is modified, wherein in step (vi) the element is Fe.

Aspects of a twenty-second embodiment are directed to a pillared silicate obtained according to the process the first through twenty-first embodiments.

Aspects of a twenty-third embodiment are directed to a pillared silicate. The pillared silicate comprises an interlayer expanded layered silicate structure, wherein the BET surface area of the pillared silicate as determined according to DIN 66131 ranges from 900 to 1,500 m²/g.

In a twenty-fourth embodiment, the pillared silicate of the twenty-second and the twenty-third embodiments is modified, wherein the BET surface area of the pillared silicate as determined according to DIN 66131 ranges from 950 to 1,450 m²/g.

In a twenty-fifth embodiment, the pillared silicate of the twenty-second through the twenty-fourth embodiments is modified, wherein the pillared silicate further comprises one or more elements supported on the pillared silicate, wherein the one or more elements are selected from the group consisting of Fe, Ru, Ir, and combinations of two or more thereof.

In a twenty-sixth embodiment, the pillared silicate of the twenty-second through the twenty-fifth embodiments is modified, wherein the one or more elements are present in an amount ranging from 0.1 to 50 wt.-% based on 100 wt-% of the pillared silicate.

In a twenty-seventh embodiment, the pillared silicate of the twenty-second through the twenty-sixth embodiments is modified, wherein the layered silicate structure comprises silicate layers selected from the group consisting of zeolite-type layers.

In a twenty-eighth embodiment, the pillared silicate of the twenty-second through the twenty-seventh embodiments is modified, wherein the layered silicate structure originates from one or more layered silicate compounds and/or is derived or derivable from one or more layered silicate compounds.

In a twenty-ninth embodiment, the pillared silicate of the twenty-second through the twenty-eighth embodiments is modified, wherein the pillared silicate is isomorphously substituted.

Aspects of a thirtieth embodiment are directed to a process for the production of one or more olefins. The process comprises the steps of: (1) providing a pillared silicate according the twenty-second through twenty-ninth embodiments; (2) contacting the pillared silicate with a gas stream comprising CO and H₂.

In a thirtieth embodiment, the process of the thirtieth embodiment is modified, wherein the contacting in step (2) is performed at a temperature ranging from 100 to 700° C.

In a thirty-second embodiment, the process of the thirtieth and the thirty-first embodiments is modified, wherein the contacting in step (2) is performed at a gas hourly space velocity of the gas stream ranging from 0.01×10⁴ to 50×10⁴ h⁻¹.

In a thirty-third embodiment, the process of the thirtieth through the thirty-second embodiments is modified, wherein the CO:H₂ molar ratio in the gas stream ranges from 0.05:1 to 1:0.05.

In a thirty-fourth embodiment, the process of the thirtieth through the thirty-third embodiments is modified, wherein the contacting in step (2) is performed at a pressure ranging from 0.01 to 20 MPa.

Aspects of a thirty-fifth embodiment are directed to a method of absorbing or ion-exchanging. The method comprises adding the pillared silicate of the twenty-second through the twenty-ninth embodiments as a molecular sieve, catalyst, catalyst component, catalyst support or binder thereof.

### BRIEF DESCRIPTION OF THE DRAWINGS

FIG. 1 depicts the X-ray diffraction pattern of a RUB-36 layered silicate as well as a RUB-36 interlayer expanded silicate and a RUB-36 pillared silicate according to one or more embodiments;

FIG. 2 depicts the nitrogen adsorption isotherms and the mesopore size distribution for a RUB-36 pillared silicate, a RUB-36 pillared silicate loaded with 8 wt.-% iron, and a RUB-36 pillared silicate loaded with 13 wt.-% iron obtained according to the examples;

FIG. 3 depicts the nitrogen adsorption isotherms and the mesopore size distribution for a RUB-36 pillared silicate, a RUB-36 pillared silicate loaded with 8 wt.-% iron, and a RUB-36 pillared silicate loaded with 13 wt.-% iron respectively obtained according to the examples;

FIG. 4 depicts the nitrogen adsorption isotherms and the mesopore size distribution for a RUB-36 pillared silicate, a RUB-36 pillared silicate loaded with 8 wt.-% iron, and a RUB-36 pillared silicate loaded with 13 wt.-% iron respectively obtained according to the examples; and

FIG. 5 depicts the X-ray diffraction patterns of a RUB-36 pillared silicate and a RUB-36 pillared silicate loaded with iron obtained according to the examples.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

One or more embodiments provide a novel process for the preparation of interlayer expanded layered silicates, i.e. pillared layered silicates. In particular, provided are novel zeolitic-type frameworks with expanded pore openings displaying improved physical and chemical properties in particular in the field of heterogeneous catalysis.

Thus, it has quite surprisingly been found that a specific pillaring of silicates may afford interlayer expanded structures with highly defined micro- and/or mesoporous frameworks displaying unprecedented physical properties in particular with respect to the surface areas which may be achieved in the resulting materials. Furthermore, it has quite unexpectedly been found that the incorporation of specific catalytically active metal species into the novel framework structures afford improved catalytic activities which clearly outperform microporous materials commonly used in the art as a support for such catalytically active metals. Even more surprisingly, however, it has been found that the interlayer expanded materials according to the present invention may accommodate novel species of the catalytically active metals and in particular metal clusters species which may not be generated in their microporous counterparts. Finally, it has quite unexpectedly been found that the inventive interlayer expanded materials, and in particular those containing specific catalytically active components and in particular specific metals, display improved catalytic activities and selectivities compared to their microporous counterparts when used as the catalytic support material.

Thus, the present invention provides a process for the production of a pillared silicate compound comprising
(i) providing a layered silicate;
(ii) interlayer expanding the layered silicate provided in step (i) comprising a step of treating the layered silicate with one or more swelling agents;
(iii) treating the interlayer expanded silicate obtained in step (ii) with one or more hydrolyzable silicon containing compounds;
(iv) treating the interlayer expanded compound obtained in step (iii) with an aqueous solution to obtain a pillared silicate;
(v) removing at least a portion of the one or more swelling agents from the pillared silicate obtained in step (iv);
(vi) impregnating the pillared silicate obtained in step (v) with one or more elements selected from the group consisting of Fe, Ru, Ir, and combinations of two or more thereof.

Concerning the layered silicate compound which is provided in step (i), there is no particular restriction according to the present invention as to the type of compound which may be used in this respect provided that it may be interlayer expanded when treated with a swelling agent in step (ii), and that it comprises chemical functionalities which under suitably chosen reaction conditions may react with a hydrolyzable silicon containing compound in steps (iv) and/or (v) for affording a pillared silicate compound.

Thus, in principle, any suitable layered silicate may be employed in the inventive process, wherein the layered silicate provided in step (i) may comprise one or more layered silicate compounds. Preferably, the one or more silicate compounds provided as layered silicate in step (i) are selected from the group consisting of MCM-22, PREFER, Nu-6(2), CDS-1, PLS-1, MCM-47, ERS-12, MCM-65, RUB-15, RUB-18, RUB-20, RUB-36, RUB-38, RUB-39, RUB-40, RUB-42, RUB-51, BLS-1, BLS-3, ZSM-52, ZSM-55, kanemite, makatite, magadiite, kenyaite, revdite, montmorillonite, and mixtures of two or more thereof. According to particularly preferred embodiments of the present invention, the layered silicate provided in step (i) comprises RUB-36 and/or RUB-39, wherein it is yet further preferred that the layered silicate provided in step (i) is RUB-36.

Layered silicates of the structure type RUB-36 are known in the art. For example, reference is made to the all-silica RUB-36 layered silicate consisting of Si and O, described in J. Song, H. Gies, Studies in Surface Science and Catalysis 2004, vol. 15, pp. 295-300, the contents of which are incorporated herein by reference. According to the present invention, the RUB-36 layered silicate is preferably defined as a compound having an X-ray diffraction pattern comprising at least the following reflections:

| Diffraction angle 2θ/° [Cu K(alpha 1)] | Intensity (%) |
|---|---|
| 7.85-8.05 | 100.0 |
| 17.04-17.24 | 1.6-5.6 |
| 20.26-20.46 | 1.7-5.7 |
| 23.89-24.09 | 4.2-12.2 |
| 24.73-24.93 | 4.8-12.8 |
| 25.30-25.50 | 2.6-6.6 |
| 26.52-26.72 | 0.7-4.7 |

wherein 100% relates to the intensity of the maximum peak in the X-ray diffraction pattern.

The precursor layered silicate RUB-39 according to the present invention is defined as a compound having having an X-ray diffraction pattern comprising at least the following reflections:

| Diffraction angle 2θ/° [Cu K(alpha 1)] | Intensity (%) |
|---|---|
| 8.15-8.35 | 100.0 |
| 16.39-16.59 | 2-12 |
| 19.87-20.07 | 7-17 |
| 23.41-23.61 | 9-19 |
| 29.94-30.14 | 1-11 |
| 35.90-36.10 | 0.5-10 |

wherein 100% relates to the intensity of the maximum peak in the X-ray diffraction pattern.

As to possible processes for the preparation of RUB-39 silicates, reference is made to WO2005/100242 A1, in particular examples 1 and 2 on pages 32 and 33; WO 2007/042531 A1, in particular example 1 on page 38, example 2 on page 39, example 3 on page 40, example 6 on page 41, and example 7 on page 42; or WO 2008/122579 A2, in particular example 1 on page 36 and example 3 on page 37.

The preparation of the layered silicate RUB-15 is disclosed, for example, in Oberhagemann, U., P. Bayat, B. Marler, H. Gies, and J. Rius in Angewandte Chemie, Intern. Ed. Engl. 1996, Vol. 35, No. 23/24, pp. 2869-2872.

As regards the preparation and characterization of the layered silicates BLS-1 and BLS-3, reference is made to WO 2010/100191 A1, the contents of which are incorporated herein by reference.

In one or more embodiments, the layered silicate provided in step (i) is isomorphously substituted. As used herein, the term “isomorphously substituted” generally refers to compounds having a two- or three-dimensional framework structure and in particular to silicate compounds or zeolitic materials containing silicon, wherein one or more of the framework elements and in particular silicon is substituted by one or more elements, as a result of which positions in the aforementioned framework which would normally be occupied by silicon are in fact occupied by one or more elements other than silicon.

According to one or more embodiments of the present invention, there is in principle no particular restriction neither with respect to the amount of the one or more elements nor with respect to the type of the one or more elements by which the layered silicate and in particular silicon contained in the layered silicate is isomorphously substituted provided that a pillared silicate may be obtained according to the inventive process. In specific embodiments, the one or more elements by which the layered silicate provided in step (i) is isomorphously substituted is selected from the group consisting of Al, B, Fe, Ti, Sn, Ga, Ge, Zr, V, Nb, Zn, Li, Be and mixtures of two or more thereof. According to very specific embodiments, the layered silicate provided in step (i) is isomorphously substituted by one or more elements selected from the group consisting of Al, B, Fe, Ti, Sn, Zr, and mixtures of two or more thereof, wherein even more specifically the one or more elements are selected from the group consisting of Al, Ti, B, and mixtures of two or more thereof. According to an embodiment, the layered silicate provided in step (i) is isomorphously substituted with Al and/or Ti, wherein in a specific embodiment the layered silicate is isomorphously substituted with Al.

Therefore, embodiments of the inventive process for the production of a pillared silicate are preferred, wherein the layered silicate provided in step (i) is isomorphously substituted, preferably with one or more elements selected from the group consisting of Al, B, Fe, Ti, Sn, Ga, Ge, Zr, V, Nb, Zn, Li, Be and mixtures of two or more thereof.

According to the inventive process, the layered silicate provided in step (i) is interlayer expanded in step (ii) using one or more swelling agents. As regards the swelling agents which may be employed in the inventive process, no particular restriction applies neither with respect to the type of the one or more swelling agents which may be used nor with respect to the amount in which they are employed provided that a suitably expanded layered silicate is obtained form the interlayer expanding step. In one or more embodiments, the swelling agents which may be used according to the present invention include surfactants and in particular cationic surfactants which when contacted with the layered silicate penetrate between the individual silicate layers in an ordered fashion for providing a swollen layered structure with an accordingly expanded interlayer spacing compared to the initial layered silicate provided in step (i). Thus, by way of example, any suitable swelling agent may be used for this purpose either alone or in combination with one or more further swelling agents.

According to specific embodiments of the inventive process, the one or more swelling agents comprise one or more compounds selected from the group consisting of cationic surfactants. In one or more embodiments, any suitable one or more cationic surfactants may be used for interlayer expanding the layered silicate. Thus, by way of example, the one or more swelling agents used in step (ii) may comprise one or more cationic surfactants selected from the group consisting of octenidine dihydrochloride, quaternary ammonium cations and salts thereof, alkyltrimethylammonium salts such as cetyl trimethylammonium bromide (CTAB; hexadecyl trimethyl ammonium bromide), cetyl trimethylammonium chloride (CTAC), cetylpyridinium chloride (CPC), polyethoxylated tallow amine (POEA), benzalkonium chloride (BAC), benzethonium chloride (BZT), 5-Bromo-5-nitro-1,3-dioxane, dimethyldioctadecylammonium chloride, dioctadecyldimethylammonium bromide (DODAB), including combinations of two or more thereof.

According to the present invention, the one or more swelling agents comprise one or more cationic surfactants containing one or more alkyl chains and preferably one or more alkyl chains having four C-atoms or more. Even more specifically, the one or more cationic surfactants contain one to four alkyl chains having four C-atoms or more, wherein even more specifically the cationic surfactant contains one to three alkyl chains having four C-atoms or more, and even more specifically one or two alkyl chains having four C-atoms or more. According to one or more embodiments wherein the one or more swelling agents comprise one or more cationic surfactants containing one or more alkyl chains having four C-atoms or more, the cationic surfactant contains one alkyl chain having four C-atoms or more. As regards the alkyl chain having four C-atoms or more in the cationic surfactant according to one or more embodiments of the present invention in this respect, there is in principle no particular restriction as to the length which said one to four alkyl chains having four C-atoms or more may have. Thus, by way of example, the one or more alkyl chains having four C-atoms or more may include one or more C₄-C₂₆ alkyl chains, wherein preferably the one or more alkyl chains include one or more C₆-C₂₄ alkyl chains, more preferably one or more C₈-C₂₂ alkyl chains, more preferably of C₁₀-C₂₀ alkyl chains, more preferably of C₁₂-C₁₈ alkyl chains, more preferably of C₁₄-C₁₈ alkyl chains, more preferably of C₁₅-C₁₇ alkyl chains. According to particularly preferred embodiments, the one or more alkyl chains having four C-atoms or more include one or more C₁₆ alkyl chains.

Therefore, according to one or more embodiments, the one or more swelling agents used in step (ii) comprise one or more compounds selected from the group consisting of cationic surfactants, wherein the one or more swelling agents comprise one or more cationic surfactants containing one or more alkyl chains having 4 C-atoms or more, wherein more specifically the cationic surfactant contains 1 to 4 alkyl chains having 4 C-atoms or more, the one or more alkyl chains having 4 C-atoms or more specifically including one or more C₄-C₂₆ alkyl chains.

According to the present invention, in one or more embodiments, the cationic surfactant comprises one or more tetraalkylammonium compounds. As regards the length of the alkyl chains of the tetraalkylammonium cations in said compounds, there is no particular restriction neither with respect to their respective length, nor with respect to their further characteristics such as whether they may be substituted and/or whether the individual alkyl moieties are respectively branched, provided that interlayer expansion of the layered silicate by action of the tetraalkylammonium compound either by itself or in combination with one or more further swelling agents may be achieved. According to a specific embodiment wherein one or more tetraalkylammonium compounds are employed, said one or more tetraalkylammonium compounds comprise one or more compounds selected from the group consisting of alkyltrimethylammonium compounds, alkylethyldimethylammonium compounds, alkyldiethylmethylammonium compounds, alkyltriethylammonium compounds, and combinations of two or more thereof. According to other embodiments, the one or more swelling agents employed in step (ii) comprise one or more tetraalkylammonium compounds selected from the group consisting of alkyltrimethylammonium compounds, alkylethyldimethylammonium compounds, and combinations of two or more thereof, wherein even more specifically one or more alkyltrimethylammonium compounds are comprised in said one or more swelling agents.

As regards the alkyltrimethylammonium compounds, alkylethyldimethylammonium compounds, alkyldiethylmethylammonium compounds, and/or alkyltriethylammonium compounds comprised in the swelling agents, there is again no particular restriction as to the alkyl group contained in said compounds, neither with respect to the chain length, nor with respect to any substitution and/or branching thereof. Thus, by way of example, the alkyl group of the aforementioned cationic surfactants comprised in the one or more swelling agents employed in step (ii), the alkyl group may be selected from the group consisting of C₄-C₂₆ alkyl chains, and more specifically from the group consisting of C₆-C₂₄ alkyl chains, more specifically of C₈-C₂₂ alkyl chains, more specifically of C₁₀-C₂₀ alkyl chains, more specifically of C₁₂-C₁₈ alkyl chains, more specifically of C₁₄-C₁₈ alkyl chains, more specifically of C₁₅-C₁₇ alkyl chains, and wherein more specifically the alkyl group is a C₁₆ alkyl chain, wherein even more specifically the one or more tetraalkylammonium compounds comprises a cetyltrimethylammonium compound.

Therefore, embodiments of the inventive process for the production of a pillared silicate are preferred, wherein the cationic surfactant comprises one or more tetraalkylammonium compounds, preferably one or more tetraalkylammonium compounds selected from the group consisting of alkyltrimethylammonium compounds, alkylethyldimethylammonium compounds, alkyldiethylmethylammonium compounds, alkyltriethylammonium compounds, and combinations of two or more thereof, wherein the alkyl group of the alkyltrimethylammonium compounds, alkylethyldimethylammonium compounds, alkyldiethylmethylammonium compounds, and/or of the alkyltriethylammonium compounds is specifically selected from the group consisting of C₄-C₂₆ alkyl chains.

Concerning the cationic surfactants which may be used in the inventive process as one or more of the swelling agents, there is equally no particular restriction as to the counterions which they may contain, provided that they allow for the swelling of the layered silicate when employed in step (ii) of the inventive process. Thus, by way of example, the counterions to the cationic surfactants according to one or more embodiments of the present invention may be selected from the group consisting of halides, hydroxides, carboxylates, nitrates, nitrites sulfates, and combinations and/or mixtures of two or more thereof, wherein it is preferred that the counterions are selected from the group consisting of fluoride, chloride, bromide, hydroxides, nitrates, and combinations and/or mixtures of two or more thereof, more preferably from the group consisting of chloride, bromide, hydroxide and combinations of two or more thereof, wherein it is particularly preferred that the counterions of the cationic surfactant according to the particularly preferred embodiments of the present invention comprise bromide.

According to an alternative embodiment of the present invention, in addition to one or more of tetraalkylammonium cations comprised in the one or more swelling agents as defined in the embodiments described in the present application, a tetraalkylammonium compound wherein the alkyl chains have three C-atoms or less, respectively, is employed in step (ii) of the inventive process. As regards the one or more tetraalkylammonium cations having three C-atoms or less said one or more tetraalkylammonium compounds are selected from the group consisting of triethyl-N-propylammonium compounds, diethyldi-N-propylammonium compounds, ethyltri-N-propylammonium compounds, tetra-N-propylammonium compounds, tetraethylammonium compounds, and combinations of two or more thereof, wherein more specifically the one or more tetraalkylammonium compounds comprise one or more tetra-N-propylammonium compounds. According to specific embodiments of the inventive process, one or more tetra-N-propylammonium compounds are further used in step (ii) together with the one or more swelling agents for the interlayer expansion of the layered silicate provided in step (i).

As regards the counterions to the one or more tetraalkylammonium compounds having alkyl chains of three C-atoms or less according to embodiments of the inventive process, there is again no particular restriction in this respect provided that interlayer expansion of the layered silicate compound may be achieved in step (ii) of the inventive process. Thus, by way of example, the counterions to said one or more tetraalkylammonium cations having alkyl chains of three C-atoms or less are selected from the group consisting of halides, hydroxide, carboxylates, nitrate, nitrite, sulfate, and combinations and/or mixtures of two or more thereof, wherein the counterions are selected from the group consisting of chloride, bromide, hydroxide, and combinations of two or more thereof, wherein even more specifically one or more tetraalkylammonium hydroxides with alkyl chains having three C-atoms or less are employed in addition to the one or more swelling agents in step (ii) of the inventive process.

Concerning the mode in which the interlayer expanding of the layered silicate is realized in step (ii) of the inventive process, there is, in principle, no particular restriction relative to the procedure which is employed to this effect nor with respect to any further agents or components which may be employed. According to one or more embodiments of the present invention, however, the interlayer expansion conducted in step (ii) is preformed in a solvent system, wherein the one or more swelling agents and according to alternative embodiments of the inventive process, the one or more tetraalkylammonium compounds having alkyl chains with three C-atoms or less are dissolved in said solvent system. According to specifically embodiments thereof, the solvent system comprises one or more solvents comprising water, wherein more specifically the solvent system preferably employed in step (ii) according to embodiments of the inventive process is water.

With respect to the further treatment of the interlayer expanded layered silicate with one or more hydrolyzable silicon containing compounds, the interlayer expanded silicate to be treated is provided in a substantially water-free state. As used herein, the term “substantially water-free” refers to a state of the interlayer expanded silicate after it has been subject to a dehydration treatment for achieving a certain state of dryness depending on the layered silicate which is employed as well as the one or more swelling agents comprised therein and on the type of duration of the dehydration treatment which is employed. Thus, there is, in principle, no particular restriction as to the type and/or duration of a dehydration treatment which may be conducted prior to step (iii) provided that a dehydrated interlayer expanded silicate may be obtained which is suited for producing a pillared silicate compound when further employed in the inventive process.

By way of example, the interlayer expanded silicate obtained in step (ii) may therefore be subject to any heating treatment for removing, i.e. for reducing the water content in said compound, and/or reduced pressure may be applied to the compound to the same effect. According to one or more embodiments of the present invention, a heat treatment is combined with applying reduced pressure to the interlayer expanded layered silicate obtained from step (ii) for dehydration thereof. As regards the heat treatment which is applied to the interlayer expanded silicate for dehydration thereof, any suitable temperature may be applied for any suitable period of time to this effect. Thus, by way of example, the interlayer expanded silicate obtained in step (ii) may be heated to a temperature in the range of from 35 to 150° C. for removing water therefrom, wherein, the interlayer expanded silicate is heated to a temperature ranging from 40 to 100° C., more specifically from 45 to 80° C., more specifically from 50 to 70° C., and more specifically from 55 to 65° C. Regarding the duration of the heat treatment, again any suitable period may be chosen for the heat treatment wherein a duration in the range of 0.25 to 24 h is utilized in one or more embodiments, and more specifically a duration ranging from 0.5 to 10 h, more specifically from 1 to 5 h, and even more specifically from 1.5 to 3 h.

Finally, concerning the application of reduced pressure to the interlayer expanded layered silicate prior to step (iii) of the inventive process, and in combination with a heat treatment according to embodiments of the present invention, there is no particular restriction relative to the vacuum applied provided that it is effective for further reducing the water content of the interlayer expanded silicate, wherein specifically vacuum in the range of anywhere from 3 kPa to 100 nPa may be applied, wherein more specifically vacuum in the range of from 1 kPa to 1 μPa is applied, more specifically of from 100 Pa to 100 μPa, more specifically of from 10 Pa to 1 mPa, and more preferably of from 1 Pa to 100 mPa.

Therefore, embodiments of the inventive process for the production of a pillared silicate compound are preferred, wherein the interlayer expanded silicate obtained in step (ii) is dehydrated prior to step (iii).

In step (iii) of the inventive process, the interlayer expanded silicate compound obtained in step (ii), which has been dehydrated subsequent to the interlayer expansion conducted in step (ii), is treated with one or more hydrolyzable silicon containing compounds. As regards the one or more hydrolyzable silicon containing compounds which may be employed according to the present invention, any suitable compounds may be employed provided that they may be hydrolyzed in the subsequent treatment step (iv) for achieving hydrolysis thereof. According to one or more embodiments of the present invention, the hydrolyzable silicon containing compound comprises one or more silicon compounds X¹X²SiX³X⁴, wherein X¹, X², X³, and X⁴ independently from one another stand for a leaving group, and in particular for a leaving group which is sensitive to hydrolysis and more specifically to hydrolysis as conducted according to particular and preferred embodiments of step (iv) of the inventive process. Thus, by way of example, the leaving group X¹, X², X³, and X⁴, which may be the same or different from one another, are selected form the group consisting of hydrogen, halogen and C₁-C₃ alkoxy, wherein more specifically the leaving group is selected from the group of hydrogen, chlorine, bromine, and C₁ and C₂ alkoxy. According to specific embodiments of the inventive process, the leaving group X¹, X², X³, and X⁴ are independently from one another selected from the group consisting of hydrogen, ethoxy, methoxy, and chlorine, wherein even more preferably the leaving group is ethoxy or methoxy. According to one embodiment thereof, the hydrolyzable silicon containing compound employed in step (iii) of the inventive process is tetraethoxysilane.

Therefore, embodiments of the inventive process for the production of a pillared silicate compound are preferred, wherein the hydrolyzable silicon containing compound comprises one or more silicon compounds X¹X²SiX³X⁴ wherein X′, X², X³, and X⁴ independently from one another stand for a leaving group, wherein the leaving group X′, X², X³, and X⁴ may be the same or different from one another.

With respect to the treatment of the interlayer expanded silicate in step (iii) with the one or more hydrolyzable silicon containing compounds, there is no particular restriction as to how the treatment is conducted provided that the treated interlayer expanded compound may afford a pillared silicate after the treatment according to step (iv) of the inventive process. Thus, any suitable means may be applied for achieving the treatment in step (iii) both with respect to the conditions used for said treatment as well as with respect to the type and amount of the one or more hydrolyzable silicon containing compounds and the amounts in which it is used. By way of example, the treatment may therefore be achieved by contacting the interlayer expanded silicate with an excess of the one or more hydrolyzable silicon containing compounds relative to the amount of said compounds which may be incorporated into the interlayer expanded silicate at most. As concerns the temperature at which said contacting may be performed, any suitable temperature may be chosen wherein temperatures comprised in the range of 25° C. to the refluxing temperature of the hydrolyzable silicon containing compound in the mixture provided in step (iii) may be chosen wherein preferably the temperature is comprised in the range of from 40° C. to the refluxing temperature, more specifically of from 50° C. to the refluxing temperature, more specifically of from 60° C. to the refluxing temperature, more specifically of from 70° C. to the refluxing temperature, wherein even more specifically the reaction in step (iii) is conducted under refluxing of the hydrolizable silicon containing compound in the mixture provided in step (iii).

Therefore, embodiments of the inventive process for the production of a pillared silicate compound are preferred, wherein in step (iii) the treatment is conducted at a temperature comprised in the range of from 25° C. to the refluxing temperature of the hydrolizable silicon containing compound in the mixture provided in step (iii).

Furthermore, as regards the duration of the treatment conducted in step (iii) of the inventive process, said duration may range anywhere from 1 to 72 h, wherein the treatment in step (iii) is conducted for a period of from 8 to 48 h, more specifically of from 12 to 36 h, more specifically of from 16 to 32 h, more specifically of from 20 to 28 h, and even more specifically of from 22 to 26 h.

For avoiding the generation of unnecessary hydrolysis products stemming from the hydrolyzable silicon containing compounds, the treated interlayer expanded compound obtained in step (iii) is preferably dried prior to the treatment in step (iv), wherein said drying treatment is performed at room temperature. In particular, said drying treatment is not performed with the aid of any heating means and/or the application of reduced pressure for avoiding that any of the hydrolyzable silicon containing compounds that are not in excess but rather contained within the interlayer expanded structure and which are the precursors to the pillaring structure obtained in step (iv) be removed from the interlayer expanded silicate treated to this effect in step (iii) of the inventive process.

After having been subject to a treatment with one or more hydrolyzable silicon containing compounds according to any one of the embodiments relative to step (iii) of the present invention, the treated interlayer expanded compound obtained therefrom is then treated with an aqueous solution for obtaining a pillared silicate. In particular, step (iv) of the inventive process allows for the hydrolysis of the one or more hydrolyzable silicon compounds as a result of which silica pillars are formed in-between the layers of the expanded structure. To this effect, any suitable aqueous solution may be employed, although it is preferred to use an aqueous solution having a pH of from 5 to 10 to this effect. According to embodiments of the inventive process, the aqueous solution used in step (iv) has a pH of from 7 to 9, and even more specifically of from 7.5 to 8.5 for achieving a suitable degree of hydrolysis of the hydrolyzable silicon containing compounds contained in the interlayer expanded silicate. For obtaining a solution displaying pH values according to specific embodiments of the inventive process, any suitable acid and/or base may be employed to this effect, wherein specifically a Bronsted acid and/or base is used to this effect. As concerns the Bronsted bases which are employed in particular for obtaining aqueous solutions of a basic pH≧7, an alkaline metal hydroxide is used to this effect, more specifically sodium and/or potassium hydroxide, and even more specifically sodium hydroxide.

As regards step (v) of the inventive process wherein at least a portion of the one or more swelling agents from the pillared silicate obtained in step (iv) is removed, any suitable means may be employed according to the present invention to this effect. Thus, by way of example, said removal may at least in part be achieved by dissolution of the swelling agent by means of using an appropriate solvent with which one or more of the swelling agents may be washed out of the pillared silicate structure. According to embodiments of the present invention wherein one or more cationic surfactants are comprised among the one or more swelling agents, said dissolution is accompanied by an ion exchange procedure performed before or during the contacting with one or more of the suitable solvents, wherein in the latter case, the ions suited for ion exchange of the one or more cationic surfactants are accordingly dissolved in the solvent used to this effect. More specifically, in cases wherein an ion exchange procedure is used for removing at least a portion of the one or more cationic surfactants, any suitable cation may be used to this effect wherein the pillared silicate compound is preferably treated with a Bronsted acid for exchanging at least part of the one or more cationic surfactants against H⁺ ions. Alternatively or in addition thereto, one or more alkaline metal containing salts may be employed which may be dissolved in the solvent which is employed for said ion exchange process for accordingly replacing at least a portion of said one or more cationic surfactants against one or more alkaline metal ions, specifically against sodium and/or potassium, and more specifically against sodium. Instead of or in combination with any one or more of the aforementioned cations which may be used for the ion exchange procedure, one or more ammonium containing salts may be employed in the ion exchange procedure for accordingly substituting at least a portion of the cationic surfactants contained in the pillared silicate compound obtained in step (iii).

According to the present invention it is however preferred that at least a portion of the one or more swelling agents is removed from the pillared silicate by degradation of said one or more swelling agents wherein said one or more swelling agents dissociate and/or decompose by any suitable physical and/or chemical means into smaller chemical compounds which may easily be removed from the pillared silicate structure either by suitable washing out thereof and/or by diffusion thereof out of the pillared silicate structure such as in the case of gaseous products formed during the degradation procedure. For achieving said degradation, any suitable means may again be employed such as degradation by solvolysis, by irradiation of the one or more swelling agents with a low wavelength source such as an ultraviolet source or higher energy radiation than ultraviolet rays, or by thermolysis, wherein according to the present invention the one or more swelling agents are removed by thermolysis.

As regards the temperature employed according to embodiments wherein the one or more swelling agents are removed by thermolysis, no particular restriction applies such that any suitable temperature may be used. Thus, by way of example, the degradation of the one or more swelling agents may be achieved in step (v) by calcination wherein the calcination temperature is comprised in the range of 250 to 850° C., more specifically from 350 to 800° C., more specifically from 400 to 750° C., more specifically from 450 to 650° C., more specifically from 500 to 600° C., and even more specifically from 525 to 575° C. According to said embodiments wherein the one or more swelling agents which are at least partly removed from the pillared silicate obtained in step (iv) is removed by calcination, the one or more swelling agents are chosen such that the calcination and in particular the calcination performed at temperatures according to the embodiments of the present invention leads to the formation of gas phase products which accordingly diffuse out of the pillared silicate structure, and in particular to the formation of gas phase products which are formed in the presence of oxygen contained in the atmosphere used for the calcination procedure.

Therefore, embodiments of the inventive process for the production of a pillared silicate are further preferred, wherein step (v) includes a calcination step for removing at least a portion of the swelling agent.

Finally, in step (vi) of the inventive process, the pillared silicate obtained in step (v) is impregnated with one or more elements selected from the group consisting of iron, ruthenium, iridium, and combinations of any two or more thereof. As regards the impregnation procedure which may be used for achieving the loading of the pillared silicate obtained in step (v) with one or more of the aforementioned elements, any suitable procedure may be used wherein one or more of the aforementioned elements is introduced into the micro- and/or mesoporous structure of the pillared silicate obtained in step (v), either as the element such as in colloidal and/or nanodisperse form or as a salt of one or more of the aforementioned elements. As regards embodiments wherein the one or more elements selected from the group consisting of Fe, Ru, Ir, and combinations of two or more thereof is introduced into the pillared silicate structure in ionic form, said one or more elements are introduced in the form of a metal complex and/or organometallic compound, and in particular in the form of a metal complex.

Therefore, according to one or more embodiments of the inventive process, the one or more sources for the one or more elements in step (vi) comprises one or more metal compounds selected from the group consisting of metal salts, metal complexes, organometallic compounds, and combinations of two or more thereof, wherein specifically the one or more metal compounds comprise one or more organometallic compounds and/or metal complexes, wherein even more specifically the one or more metal compounds comprise one or more metal complexes.

As regards the metal salts which are employed as the one or more sources of the one or more elements in step (vi) according to particular embodiments of the inventive process, any suitable salt or salt mixtures may be employed provided that they may be effectively impregnated into the pillared silicate structure. Thus, by way of example, said one or more metal salts may comprise one or more compounds selected form the group consisting metal halides, metal hydroxides, metal carbonates, metal carboxylates, metal nitrates, metal nitrites, metal phosphates, metal phosphites, metal phosphonates, metal phosphinates, metal sulfates, metal sulfites, metal sulfonates, metal alkoxides, and combinations of two or more thereof, preferably from the group consisting of metal halides, metal nitrates, metal nitrites, metal sulfates, metal sulfites, and combinations of two or more thereof, more specifically from the group consisting of metal halides, metal nitrates, metal sulfates, and combinations of two or more thereof, more specifically from the group consisting of metal nitrates and/or metal sulfates, wherein more specifically the metal salts comprise one or more metal nitrates.

As regards the embodiments of the present invention wherein the one or more sources for the one or more elements in step (vi) comprise one or more metal complexes, there is no particular restriction according to the inventive process as to the ligands contained in the metal complexes such that, by way of example, these may be selected from the group consisting of mono-, bi-, tri-, tetra-, penta-, and hexadentate ligands, including combinations of any two or more thereof. According to embodiments thereof, the ligands of the metal complexes are selected from the group consisting of halide, pseudohalide, H₂O, NH₃, CO, hydroxide, oxalate, ethylenediamine, 2,2′-bipyridine, 1,10-phenanthroline, acetylacetonate, 2,2,2-crypt, diethylenetriamine, dimethylglyoximate, EDTA, ethylenediaminetriacetate, glycinate, triethylenetetramine, tris(2-aminoethyl)amine, and combinations of two or more thereof, more preferably from the group consisting of fluoride, chloride, bromide, cyanide, cyanate, thiocyanate, NH₃, CO, hydroxide, oxalate, ethylenediamine, acetylacetonate, diethylenetriamine, dimethylglyoximate, EDTA, ethylenediaminetriacetate, glycinate, triethylenetetramine, tris(2-aminoethyl)amine, and combinations of two or more thereof, more specifically from the group consisting of chloride, bromide, cyanide, NH₃, CO, oxalate, ethylenediamine, acetylacetonate, diethylenetriamine, EDTA, thylenediaminetriacetate, triethylenetetramine, and combinations of two or more thereof, more specifically from the group consisting of cyanide, ethylenediamine, acetylacetonate, diethylenetriamine, EDTA, ethylenediaminetriacetate, triethylenetetramine, and combinations of two or more thereof, wherein more specifically the complex ligand is cyanide, and wherein even more specifically the metal complex comprises hexacyanoferrate, preferably hexacyanoferrate(II).

With respect to the impregnation procedure employed in step (vi), any suitable impregnation means may be employed involving the use of any suitable solvent system to this effect depending on the type and amount of the one or more sources for the one or more elements selected from the group consisting of Fe, Ru, Ir, and combinations of two or more thereof. Thus, by way of example, the solvent system employed for the impregnation procedure is not particularly restricted provided that the dissolution and/or suspension of the one or more sources for the one or more elements may be achieved for the impregnation process. Thus, in the event that the one or more sources of the one or more elements is used in the form of one or more metal complexes and/or organometallic compounds which are charge neutral and/or which may be dissolved without solvation of at least part of the one or more metal compounds, any suitable solvent system may be employed to this effect wherein specifically a solvent system comprising one or more organic solvents may be used to this effect.

As regards the organic solvents which may be employed according to said embodiments of the present invention, these may be selected among non-polar solvents, polar aprotic solvents, and polar protic solvents, including mixtures of two or more thereof, wherein the non-polar solvents may for example be selected from the group consisting of pentane, cyclopentane, hexane, cyclohexane, benzene, toluene, 1,4-dioxane, chloroform, and diethylether. The polar aprotic solvents may be for example selected from the group consisting of dichloromethane, tetrahydrofurane, ethylacetate, acetone, dimethylformamide, acidonitrile, dimethylsulfoxide, and propylenecarbonate. Finally, as polar protic organic solvent any one or more of formic acid, N-butanol, isopropanol, N-propanol, ethanol, methanol, and acidic acid may be used to this effect. As regards embodiments of the present invention wherein the one or more sources for the one or more elements in step (vi) comprise one or more metal salts which are impregnated into the pillared silicate in their dissociated, i.e. in the solvated state, the solvent system used to this effect comprises a polar protic solvent which may for example be selected from the group consisting of N-propanol, ethanol, methanol, acidic acid, and water including mixtures of two or more thereof, wherein according to said embodiments of the inventive process the solvent system comprises ethanol, methanol, water, or mixtures of two or more thereof, and more specifically comprises water, wherein said cases that water is used as the solvent system for the impregnation in step (vi) of the inventive process.

Thus, concerning the means of impregnation employed in step (vi) of the inventive process, again any suitable means may again be used to this effect such as by contacting the pillared silicate obtained in step (v) with a solvent system comprising the one or more sources of the one or more elements selected from the group consisting of Fe, Ru, Ir, and combinations of two or more thereof defined in the present application. Said contacting may be performed such that an excess of the solvent system containing the one or more sources for the one or more elements is employed in excess for achieving the impregnation of the pillared silicate which is then filtered off from the solution and optionally dried and/or calcinated. According to an alternative embodiment of the present invention, however, the impregnation in step (vi) is achieved by incipient wetness such that the pillared silicate obtained in step (v) is treated with a specific amount of a solvent system comprising the one or more sources for the one or more elements allowing for complete absorption thereof into the micro- and/or mesoporous system of the pillared silicate.

As regards embodiments of the inventive process wherein the one or more elements impregnated into the pillared silicate in step (vi) are employed in the form of a metal compound and in particular in the form of a metal complex and/or an organometallic compound, the impregnated silicate is subject to a further step for transforming the one or more metal compounds into the metal and/or into a metal oxide of the one or more elements selected from the group consisting of Fr, Ru, Ir, including alloys and/or mixed oxides of any two or more thereof. As for the degradation of the one or more swelling agents in step (v), any suitable means may be employed for degrading the counterions and/or ligands and/or organometallic moieties wherein the particular means relative to the degradation of the one or more swelling agents as defined in the present application apply accordingly with respect to the degradation of the aforementioned counterions, ligands, and/or organometallic moieties, i.e. organic moieties bound to the metal in the organometallic compounds. In particular, according to said embodiments, the impregnated pillared silicate obtained in step (vi) is subject to a degradation procedure and, in particular, to a calcination procedure according to any of the particular embodiments relative to the degradation of the one or more swelling agents for obtaining a pillared silicate loaded with one or more elements selected from the group consisting of Fe, Ru, Ir, and combinations of two or more thereof, wherein said one or more elements are, at least in part, present in the pillared silicate as the metal and/or as an oxide of said metal or metals, wherein in the event that two or more metals have been impregnated into the pillared silicate in step (vi), two or more of said two or more elements may be contained in the pillared silicate in the form of an alloy and/or a mixed oxide of two or more of said two or more elements.

As regards the workup of the impregnated pillared silicate obtained in step (vi), any suitable one or more steps may be conducted which according to particular embodiments include a step of calcining the impregnated pillared silicate. Thus, by way of example, the impregnated pillared silicate obtained in step (vi) may be subject to any number and/or sequence of washing and/or drying steps preferably prior to calcination thereof according to the particular embodiments of the inventive process including said step. It is, however, preferred according to the present invention that the pillared silicate obtained in step (vi) of the inventive process is simply dried without having been subject to a washing step for avoiding the loss of any portion of the one or more elements which have been impregnated into the pillared silicate in step (vi), in particular in instances wherein the washing solvent system would lead to the dissolution of at least part of the one or more sources for the one or more elements employed in step (vi).

Therefore, according to embodiments of the inventive process, the production of a pillared silicate further comprises
(vii) drying the impregnated pillared silicate; and/or
(viii) calcining the impregnated and optionally dried pillared silicate.

As regards the calcination conducted in step (viii) of the inventive process, as for the calcination conducted in step (v), there is no particular restriction as to the temperature which may be employed therein, wherein it is preferred that said calcination is conducted at a temperature ranging from 400 to 950° C., specifically from 450 to 850° C., more specifically from 500 to 750° C., more specifically from 550 to 700° C., and even more specifically from 600 to 650° C.

As regards the optional workup of the impregnated pillared silicate obtained in step (vi), said optional steps may be repeated once or several times for improving the quality of the resulting material. Furthermore, depending on the desired loading of the pillared silicate, the impregnation in step (vi) may be repeated once or several times to this effect. Thus, according to embodiments of the inventive process, the impregnating of the pillared silicate obtained in step (v) with Fe, Ru, Ir, or mixtures of two or more thereof in step (vi) is repeated one or more times and specifically one to three times, more specifically once or twice, and even more specifically once. According to said embodiments, in addition to repeating step (vi) one or more times, also step (vii) of drying the respectively impregnated silicate is repeated one or more times such that it is preferred that steps (vi) and (vii) are repeated one or more times, specifically one to three times, more specifically once or twice and even more specifically once. According to said embodiments, it is further preferred that step (viii) of calcining the respectively impregnated and dried pillared silicate is equally repeated one or more times such that according to said further embodiments, steps (vi) to (vii) are repeated one or more times, specifically one to three times, more specifically once or twice, and even more specifically once.

Therefore, according to embodiments of the inventive process, step (vi), specifically steps (vi) and (vii), and more specifically steps (vi) to (viii) are repeated one or more times, specifically one to three times, more specifically once or twice, and even more specifically once.

After the optional workup of the impregnated pillared silicate obtained in step (vi) of the inventive process and in particular after calcination thereof in step (viii) as defined according to particular embodiments of the inventive process, it is further preferred in addition to or alternative thereto that the pillared silicate is treated with a reducing agent.

Therefore, according to embodiments of the present invention, the process for the production of a pillared silicate further comprises
(ix) treating the impregnated and optionally dried and/or optionally calcined pillared silicate with a reducing agent.

As regards the reducing agent which may be employed in step (ix), no particular restriction applies according to the present invention such that any suitable reducing agent may be employed provided that at least a portion of the one or more element selected from the group consisting of Fe, Ru, Ir, and combinations of two or more thereof impregnated into the pillared silicate may effectively be reduced by the use thereof. Thus, by way of example, the reducing agent used in step (ix) may comprise one or more compounds selected from the group consisting of hydrogen, hydrides, metals, sulfites, phosphites, hypophosphites, hydrazine, and combinations of two or more thereof, more preferably from the group consisting of hydrogen, LiAlH₄, diisobultyaluminum hydride, NaBH₄, Sn(II) salts, Fe(II) salts, sulfites, phosphites, hypophosphites, hydrazine, and combinations of two or more thereof, more preferably from the group consisting of hydrogen, NaBH₄, Sn(II) chloride, Fe(II) sulfate, hypophosphites, and combinations of two or more thereof, wherein even more preferably the reducing agent is hydrogen.

According to embodiments of the present invention, no calcination step (viii) is performed prior to step (ix) of treating the optionally dried pillared silicate with a reducing agent. According to said embodiments, the impregnated pillared silicate obtained in step (vi) is therefore directly subject to treatment with a reducing agent, wherein specifically said treatment is achieved using a reducing agent in the gas form, wherein in particular reduction is performed at a temperature ranging anywhere from 100 to 600° C. with a gaseous reducing agent. More specifically, reduction in step (ix) is performed at a temperature ranging from 150 to 550° C. according to said embodiments, and more specifically from 200 to 520° C., more specifically from 250 to 500° C., more specifically from 300 to 480° C., more specifically from 350 to 450° C., more specifically from 380 to 430° C., more specifically from 390 to 410° C. According to said embodiments, there is no particular restriction as to the gaseous reducing agent which may be used, provided that at least a portion of the one or more elements impregnated into the pillared silicate in step (vi) may be reduced, wherein preferably H₂ is used as the gaseous reducing agent.

According to the present invention, there is no particular preference relative to the one or more elements selected from the group consisting of Fe, Ru, Ir, and any combinations of two or more thereof which are chosen for impregnation into the pillared silicate in step (vi). According to embodiments of the present invention, the pillared silicate obtained in step (v) is impregnated with one or more elements including iron, wherein it is yet further preferred that only iron is impregnated into the pillared silicate in step (vi) of the inventive process.

Therefore, embodiments of the inventive process are particularly preferred, wherein in step (vi) the element is Fe.

In addition to the process for the production of a pillared silicate according to embodiments defined in the present application, the present invention further relates to a pillared silicate which is obtainable according to the inventive process for the production of a pillared silicate including any one of the embodiments thereof. In particular, the present invention relates to pillared silicates which may either be obtained according to any one of the embodiments of the inventive process, as well as to a pillared silicate which may be obtained according to any of the aforementioned embodiments, but which however have been obtained according to a different process than the one defined in the present application.

Furthermore, the present invention also relates to a pillared silicate per se comprising an interlayer expanded layered silicate structure, wherein the BET surface area of the pillared silicate as determined according to DIN 66131 ranges from 900 to 1500 m²/g. Although the aforementioned pillared silicate according to the present invention may be obtained according to any conceivable method, it is preferred that said pillared silicate is a pillared silicate obtainable according to the inventive process as defined in any one of the embodiments, and even more specifically is a pillared silicate which is obtained according to any one of said embodiments of the inventive process.

As regards the BET surface area of the pillared silicate, it has quite surprisingly been found that a pillared silicate may be provided according to the present invention which displays far higher surface areas than those which may be obtained according to the teaching of the prior art. More specifically, the BET surface area of the pillared silicate according to the present invention as determined according to DIN 66131 may range from 950 to 1,450 m²/g, wherein more specifically, the surface area of the pillared silicate ranges from 1,000 to 1,400 m²/g, more specifically from 1,050 to 1,350 m²/g, more specifically from 1,100 to 1,300 m²/g, and even more specifically from 1,150 to 1,250 m²/g.

As regards the pillared silicate according to the present invention, which is defined by a specific surface area, it is noted that said embodiment of the present invention wherein said pillared silicate is obtainable according to the inventive process does not necessarily involve either of steps (v) or (vi) for being obtained. Thus, according to said embodiments of the present invention, the pillared silicate having a BET surface area as determined according to DIN 66131 ranging from 900 to 1,500 m²/g is obtainable according to a process comprising steps (i) to (iv) and more preferably comprising steps (i) to (v) of the inventive process wherein same applies with respect to embodiments of the individual steps (i) to (iv) and preferably steps (i) to (v) as defined in the present application relative to the corresponding steps in particular embodiments of the inventive process for the production of a pillared silicate.

Nevertheless, it is preferred that, as defined in the foregoing, the pillared silicate of the present invention displaying a specific surface area as determined according to DIN 66131 ranging from 900 to 1,500 m²/g is obtainable and is obtained according to any of the embodiments of the inventive process.

Thus, according to one or more embodiments of the invention, the pillared silicate additionally contains one or more elements supported on the pillared silicate. In this respect, no particular restriction applies relative to the one or more elements which may be supported thereon, wherein it is preferred that said one or more elements comprise one or more transition metal elements, and more specifically one or more elements selected form the group consisting of Sn, Ti, Mo, Mn, Fe, Co, Cu, Zn, Zr, Ru, Pd, Ag, Pt, Au, Ir, Rh, Sm, Eu, and combinations of two or more thereof, and more specifically from the group consisting of Ti, Mo, Fe, Co, Cu, Ru, Pd, Ag, Pt, Au, Ir, Rh, and combinations of two or more thereof. According to embodiments of the inventive pillared silicate, the one or more elements supported on the pillared silicate are selected from the group consisting of Fe, Ru, Ir, and combinations of two or more thereof, wherein even more specifically the pillared silicate further contains Fe which is supported thereon.

Therefore, embodiments of the inventive pillared silicate are preferred, wherein the pillared silicate additionally contains one or more elements supported on the pillared silicate.

As regards the one or more elements additionally contained in the inventive pillared silicate and supported thereon, there is no particular restriction according to the present invention neither regarding their number nor with respect to the amount in which the one or more elements may be contained in the pillared silicate. Thus, by way of example, the one or more elements additionally contained and supported on the pillared silicate according to any of the particular embodiments of the present invention may be present in an amount ranging anywhere from 0.1 to to 50 wt.-% based on 100 wt-% of the pillared silicate, wherein the one or more elements are contained therein in an amount ranging from 0.5 to 30 wt.-%, more specifically from 1 to 25 wt.-%, more specifically from 3 to 20 wt.-%, more specifically from 5 to 16 wt.-%, more specifically from 6 to 15 wt.-%, more specifically from 7 to 14 wt.-%, and even more specifically from 8 to 13 wt.-%.

Therefore, embodiments of the inventive pillared silicate are preferred, wherein the pillared silicate contains the one or more elements in an amount ranging from 0.1 to 50 wt.-% based on 100 wt-% of the pillared silicate.

According to the present invention, the pillared silicate compound may comprise any type of silicate layers, provided that said layers are suited for forming a pillared silicate structure according to particular embodiments of the present invention. As regards the term “layered silicate structure” as used in the present invention, said term generally refers to a structure comprising a regular array of silicate sheet layers which are stacked in parallel. In the present invention, the layers contained in said layered silicate structures are accordingly referred to a “silicate layers”. Thus, as used herein, a layered silicate structure may refer to such arrangements of silicate layers as may be found in phyllosilicates or also to regular stackings of silicate layers as may be found in layer-based zeolite structures and layered precursors of such layer-based zeolite structures. According to preferred embodiments, of the present invention, wherein the silicate layers comprise layers as may be found in a zeolite structure and/or in precursor compounds to layer-based zeolite structures, it is further preferred that said silicate layers are selected from the group consisting of zeolite-type layers. In general, such zeolite-type layers may be selected from any conceivable type of zeolite structure provided that these are suitable for forming a layered silicate structure and may form a pillared silicate according to any of the particular and preferred embodiments of the present invention. According to particularly preferred embodiments, the layered silicate structure comprises zeolite-type layers selected from the group consisting of HEU-type layers, FER-type layers, MWW-type layers, RWR-type layers, CAS-type layers, SOD-type layers, RRO-type layers, or combinations of two or more different types of these zeolite-type layers, wherein even more preferably the layered silicate structure has FER-type layers.

Therefore, embodiments of the inventive pillared silicate are further preferred, wherein the layered silicate structure comprises silicate layers selected from the group consisting of zeolite-type layers.

According to further embodiments of the present invention, the layered silicate structure of the pillared silicate actually originates from one or more layered silicate compounds, preferably from a layered silicate compound which has been used in its production. In general, the term “layered silicate compound” as employed in the present invention designates any natural or synthetic layered silicate, wherein preferably said term refers to layered silicates employed as a catalyst and/or a catalyst substrate in industrial processes. Alternatively or in addition thereto, the layered silicate structure is preferably derivable from one or more silicate compounds. Regarding said layered silicate compounds from which the layered silicate structure of the pillared silicate compound preferably originates and/or may be derived from, these include one or more of any conceivable layered silicate compound provided that it is suitable for forming a pillared silicate according to any of the particular or preferred embodiments of the present invention. According to preferred embodiments of the present invention, the one or more layered silicate compounds comprise one or more zeolites. Within the meaning of the present invention, layered silicate compounds from which the layered silicate structure of the pillared silicate is preferably derived or derivable from includes derivatives of a layered silicate compound, wherein a derivative of a layered silicate compound generally refers to a layered silicate compound which has been subject to one or more physical and/or chemical modifications, preferably to one or more chemical and/or physical modification for improving its suitability to allow for a pillaring thereof according to any of the particular or preferred embodiments of the present invention.

According to specific embodiments of the present invention, said one or more layered silicate compounds comprise one or more layered silicates selected from the group consisting of MCM-22, PREFER, Nu-6(2), CDS-1, PLS-1, MCM-47, ERS-12, MCM-65, RUB-15, RUB-18, RUB-20, RUB-36, RUB-38, RUB-39, RUB-40, RUB-42, RUB-51, BLS-1, BLS-3, ZSM-52, ZSM-55, kanemite, makatite, magadiite, kenyaite, revdite, montmorillonite, and combinations of two or more thereof, wherein the one or more layered silicate compounds preferably comprise RUB-36 and/or RUB-39, and even more preferably wherein the layered silicate structure originates from RUB-36.

Therefore, embodiments of the inventive pillared silicate are yet further preferred, wherein the layered silicate structure originates from one or more layered silicate compounds and/or is derived or derivable from one or more layered silicate compounds.

Regarding the specific layered silicate compounds defined in the foregoing, RUB-18 refers to specific layered silicates of which the preparation is, for example, described in T. Ikeda, Y. Oumi, T. Takeoka, T. Yokoyama, T. Sano, and T. Hanaoka Microporous and Mesoporous Materials 2008, 110, pp. 488-500. RUB-20 refers to specific layered silicates which may be prepared as, for example, disclosed in Z. Li, B. Marler, and H. Gies Chem. Mater. 2008, 20, pp. 1896-1901. RUB-51 refers to specific layered silicates of which the preparation is, for example, described in Z. Li, B. Marler, and H. Gies Chem. Mater. 2008, 20, pp. 1896-1901. ZSM-52 and ZSM-55 refer to specific layered silicates which may be prepared as, for example, described in D. L. Dorset, and G. J. Kennedy J. Phys. Chem. B. 2004, 108, pp. 15216-15222. Finally, RUB-38, RUB-40, and RUB-42 respectively refer to specific layered silicates as, for example, presented in the presentation of B. Marler and H. Gies at the 15th International Zeolite Conference held in Beijing, China in August 2007.

According to specific embodiments of the present invention, the silicate layers of the layered silicate structure comprised in the pillared silicate compound are isomorphously substituted with one or more types of heteroatoms. In general, in said specific embodiments, any conceivable type of heteroatom may isomorphously substitute at least a portion of the Si atoms in the silicate structure of the silicate layers, provided that the one or more types of heteroatoms are suitable for isomorphous substitution. In particular, it is preferred that the silicate layers are isomorphously substituted with one or more elements selected from the group consisting of Al, B, Fe, Ti, Sn, Ga, Ge, Zr, V, Nb, Zn, Li, Be and mixtures of two or more thereof, more preferably from the group consisting of Al, B, Fe, Ti, Sn, Zr, and mixtures of two or more thereof, more preferably from the group consisting of Al, Ti, B, and mixtures of two or more thereof, and wherein even more preferably, the silicate structure is isomorphously substituted with Al and/or Ti.

Therefore, embodiments of the inventive pillared silicate are yet further preferred, wherein the pillared silicate, and preferably the layered silicate structure of the pillared silicate, is isomorphously substituted.

In addition to the process for the production of a pillared silicate, the present invention further relates to a process for the production of one or more olefins comprising the steps of:
(1) providing a pillared silicate according to the present invention including any of the particular and preferred embodiments as defined in the present application;
(2) contacting the pillared silicate with a gas stream comprising CO and H₂.

As regards the form in which the pillared silicate may be provided in the inventive process for the production of one or more olefins, no particular restriction applies according to the present invention, provided that one or more olefins may be produced in step (2) upon contacting thereof with a gas stream comprising CO and H₂. Thus, by way of example, the pillared silicate may be provided as such or in the form of a molding or extrudate, i.e. as a shaped body which has preferably been formed with the aid of a binder.

Furthermore, no particular restriction applies relative to the mode in which the inventive process for the production of one or more olefins is conducted, such that in principle both a batch-process as well as a continuous process may be chosen. According to embodiments of the present invention, however, the process for the production of one or more olefins is conducted at least in part as a continuous process.

As regards the conditions under which the pillared silicate is contacted with a gas stream comprising CO and H₂ in step (2) of the inventive process, again, no particular restriction applies in this respect provided that one or more olefins may be generated by said contacting. Thus, as regards the temperature at which said contacting is performed, said temperature may range anywhere from 100 to 700° C., wherein specifically the temperature ranges from 150 to 500° C., more specifically from 200 to 400° C., and even more specifically from 250 to 350° C. According to an embodiment of the inventive process the temperature at which the pillared silicate is contacted with a gas stream comprising CO and H₂ ranges from 280 to 320° C.

Same applies accordingly with respect to the gas hourly space velocity (GHSV) of the gas stream comprising CO and H₂ which may for example range anywhere from 0.01×10⁴ to 50×10⁴ h⁻¹, and preferably ranges from 0.05×10⁴ to 20×10⁴ h⁻¹, more specifically from 0.1×10⁴ to 10×10⁴ h⁻¹, more specifically from 0.2×10⁴ to 5×10⁴ h⁻¹, more specifically from 0.5×10⁴ to 2.5×10⁴ h⁻¹, more specifically from 0.7×10⁴ to 2.0×10⁴ h⁻¹, more specifically from 0.9×10⁴ to 1.8×10⁴ h⁻¹, and even more specifically from 1×10⁴ to 1.5×10⁴ h⁻¹.

Regarding the composition of the gas stream comprising CO and H₂ which is contacted in step (2) of the inventive process, said gas stream may comprise one or more additional gases in addition to CO and H₂ such as CO₂ and/or H₂O and/or one or more inert gases including N₂. According to embodiments of the present invention, however, the gas stream does not comprise any substantial amount of a further gas in addition to CO and H₂. As regards the amounts of CO and H₂ which are comprised in the gas stream for contacting with the pillared silicate, no particular restriction applies in this respect provided that at least part of the gas stream may react to one or more olefins upon contacting the pillared silicate in step (2). Thus, by way of example, the CO:H₂ molar ratio in the gas stream may range anywhere from 0.05:1 to 1:0.05, wherein preferably the molar ratio ranges from 0.1:1 to 1:0.1, more preferably from 0.3:1 to 1:0.3, more preferably from 0.6:1 to 1:0.6, more preferably from 0.8:1 to 1:0.8, and even more preferably from 0.9:1 to 1:0.9.

Finally, as regards the pressure at which the contacting in step (2) of the inventive process is performed, again no particular restriction applies in this respect provided that one or more olefins may be produced in the inventive process. Thus, the contacting in step (2) of the inventive process may range anywhere from 0.01 to 20 MPa, wherein it is preferred that said contacting is performed at a pressure ranging from 0.05 to 5 MPa, and more specifically from 0.1 to 2 MPa. According to embodiments of the present invention, the contacting in step (2) of the gas stream comprising CO and H₂ with the pillared silicate is performed at a pressure greater than atmospheric pressure and in particular at a pressure ranging from 0.3 to 1 MPa, more preferably from 0.4 to 0.7 MPa, wherein according to embodiments of the present invention, contacting in step (2) is performed at a pressure ranging from 0.45 to 0.55 MPa.

Finally, the present invention relates to the use of a pillared silicate compound as defined in the foregoing according to any of the embodiments described in the present application in any suitable application, wherein it is preferred that the inventive pillared silicate is employed as a molecular sieve, catalyst, catalyst component, catalyst support or binder thereof, as adsorbents, and/or for ion exchange.

The present invention includes the following embodiments, wherein these include the specific combinations of embodiments as indicated by the respective interdependencies defined therein:
1. A process for the production of a pillared silicate comprising:
   (i) providing a layered silicate;
   (ii) interlayer expanding the layered silicate provided in step (i) comprising a step of treating the layered silicate with one or more swelling agents;
   (iii) treating the interlayer expanded silicate obtained in step (ii) with one or more hydrolyzable silicon containing compounds;
   (iv) treating the interlayer expanded compound obtained in step (iii) with an aqueous solution to obtain a pillared silicate;
   (v) removing at least a portion of the one or more swelling agents from the pillared silicate obtained in step (iv);
   (vi) impregnating the pillared silicate obtained in step (v) with one or more elements selected from the group consisting of Fe, Ru, Ir, and combinations of two or more thereof.
2. The process of embodiment 1, further comprising:
   (vii) drying the impregnated pillared silicate; and/or
   (viii) calcining the impregnated and optionally dried pillared silicate.
3. The process of embodiment 1 or 2, further comprising:
   (ix) treating the impregnated and optionally dried and/or optionally calcined pillared silicate with a reducing agent.
4. The process of any of embodiments 1 to 3, wherein the interlayer expanded silicate obtained in step (ii) is dehydrated prior to step (iii).
5. The process of any of embodiments 1 to 4, wherein the layered silicate provided in step (i) comprises one or more layered silicate compounds selected from the group consisting of MCM-22, PREFER, Nu-6(2), CDS-1, PLS-1, MCM-47, ERS-12, MCM-65, RUB-15, RUB-18, RUB-20, RUB-36, RUB-38, RUB-39, RUB-40, RUB-42, RUB-51, BLS-1, BLS-3, ZSM-52, ZSM-55, kanemite, makatite, magadiite, kenyaite, revdite, montmorillonite, and combinations of two or more thereof.
6. The process of any of embodiments 1 to 5, wherein the layered silicate provided in step (i) is isomorphously substituted.
7. The process of any of embodiments 1 to 6, wherein the one or more swelling agents used in step (ii) comprise one or more compounds selected from the group consisting of cationic surfactants.
8. The process of embodiment 7, wherein the cationic surfactant contains 1 to 4 alkyl chains having 4 C-atoms or more.
9. The process of embodiment 7 or 8, wherein the one or more alkyl chains having 4 C-atoms or more include one or more C₄-C₂₆ alkyl chains.
10. The process of any of embodiments 7 to 9, wherein the cationic surfactant comprises one or more tetraalkylammonium compounds.
11. The process of any of embodiments 1 to 10, wherein the hydrolyzable silicon containing compound comprises one or more silicon compounds X¹X²SiX³X⁴ wherein X¹, X², X³, and X⁴ independently from one another stand for a leaving group, wherein the leaving group X¹, X², X³, and X⁴ may be the same or different from one another.
12. The process of any of embodiments 1 to 11, wherein in step (iii) the treatment is conducted at a temperature comprised in the range of from 25° C. to the refluxing temperature of the hydrolizable silicon containing compound in the mixture provided in step (iii).
13. The process of any of embodiments 1 to 12, wherein in step (iii) the treatment is conducted for a period of from 1 to 72 h.
14. The process of any of embodiments 1 to 13, wherein the aqueous solution used in step (iv) has a pH of from 5 to 10.
15. The process of any of embodiments 1 to 14, wherein step (v) includes a calcination step for removing at least a portion of the swelling agen.
16. The process of any of embodiments 1 to 15, wherein the calcination in step (viii) is conducted at a temperature ranging from 400 to 950° C.
17. The process of any of embodiments 1 to 16, wherein the one or more sources for the one or more elements in step (vi) comprise one or more metal compounds selected from the group consisting of metal salts, metal complexes, organometallic compounds, and combinations of two or more thereof.
18. The process of embodiment 17, wherein the ligands of the metal complexes are selected from the group consisting of mono-, bi-, tri-, tetra-, penta-, and hexadentate ligands, including combinations of two or more thereof.
19. The process of any of embodiments 1 to 18, wherein step (vi) is repeated one or more times.
20. The process of any of embodiments 1 to 19, wherein the reducing agent in step (ix) comprises one or more compounds selected from the group consisting of hydrogen, hydrides, metals, sulfites, phosphites, hypophosphites, hydrazine, and combinations of two or more thereof.
21. The process of any of embodiments 1 to 20, wherein in step (vi) the element is Fe.
22. A pillared silicate obtainable according to the process of any one of embodiments 1 to 21.
23. A pillared silicate comprising an interlayer expanded layered silicate structure, wherein the BET surface area of the pillared silicate as determined according to DIN 66131 ranges from 900 to 1,500 m²/g.
24. The pillared silicate of embodiment 22 or 23, wherein the BET surface area of the pillared silicate as determined according to DIN 66131 ranges from 950 to 1,450 m²/g.
25. The pillared silicate of any of embodiments 22 to 24, wherein the pillared silicate additionally contains one or more elements supported on the pillared silicate, wherein the one or more elements are selected from the group consisting of Fe, Ru, Ir, and combinations of two or more thereof.
26. The pillared silicate of embodiment 25, wherein the pillared silicate contains the one or more elements in an amount ranging from 0.1 to 50 wt.-% based on 100 wt-% of the pillared silicate.
27. The pillared silicate of any of embodiments 22 to 26, wherein the layered silicate structure comprises silicate layers selected from the group consisting of zeolite-type layers.
28. The pillared silicate of any of embodiments 22 to 27, wherein the layered silicate structure originates from one or more layered silicate compounds and/or is derived or derivable from one or more layered silicate compounds.
29. The pillared silicate of any of embodiments 22 to 28, wherein the pillared silicate is isomorphously substituted.
30. A process for the production of one or more olefins comprising the steps of:
   (1) providing a pillared silicate according to any of embodiments 22 to 29;
   (2) contacting the pillared silicate with a gas stream comprising CO and H₂.
31. The process of embodiment 30, wherein the contacting in step (2) is performed at a temperature ranging from 100 to 700° C.
32. The process of embodiment 30 or 31, wherein the contacting in step (2) is performed at a gas hourly space velocity of the gas stream ranging from 0.01×10⁴ to 50×10⁴ h⁻¹.
33. The process of any of embodiments 30 to 32, wherein the CO:H₂ molar ratio in the gas stream ranges from 0.05:1 to 1:0.05.
34. The process of any of embodiments 30 to 33, wherein the contacting in step (2) is performed at a pressure ranging from 0.01 to 20 MPa.
35. Use of a pillared silicate according to any of embodiments 22 to 29 as a molecular sieve, catalyst, catalyst component, catalyst support or binder thereof, as absorbents, and/or for ion-exchange.

The present invention is explained in more detail with reference to the examples and figures described below.

### DESCRIPTION OF THE FIGURES

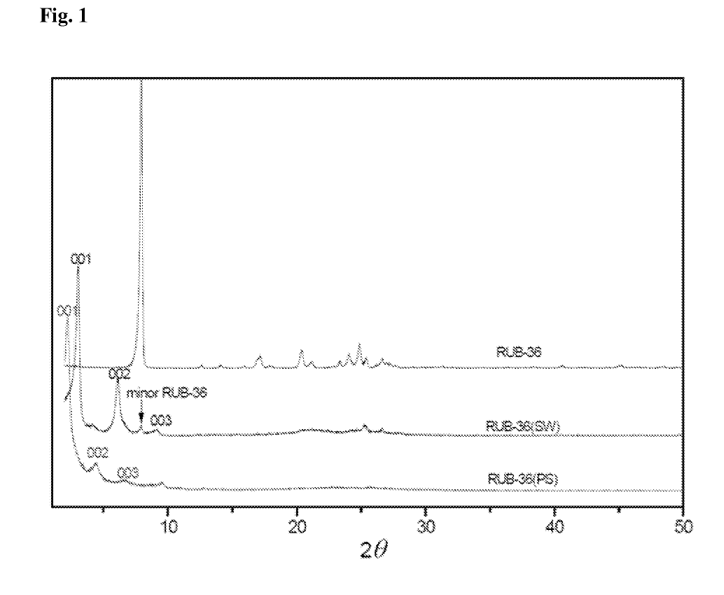

FIG. 1 displays the X-ray diffraction pattern of the RUB-36 layered silicate used as starting material in Examples 1 and 2 as well as the RUB-36 interlayer expanded silicate and the RUB-36 pillared silicate respectively obtained according to Example 1. In the figure, the diffraction angle in ° 2Theta is designated as “2θ” and is plotted along the abscissa, and the diffraction intensity in arbitrary units is plotted along the ordinate. In the Figure, the diffraction patterns of RUB-36 interlayer expanded silicate is designated as “RUB-36(SW)”, whereas the diffraction patterns of RUB-36 pillared silicate is designated as “RUB-36(PS)”.

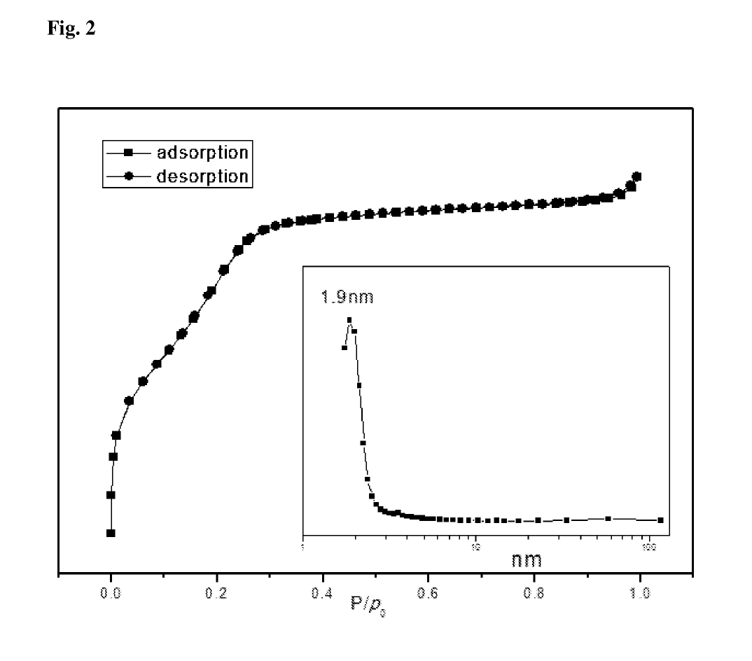

FIGS. 2-4 show both the nitrogen adsorption isotherms and the mesopore size distribution for the RUB-36 pillared silicate, the RUB-36 pillared silicate loaded with 8 wt.-% iron, and the RUB-36 pillared silicate loaded with 13 wt.-% iron respectively obtained according to Examples 1, 3 and 4. In the figures, the relative pressure “P/p₀” is plotted along the abscissa for the nitrogen adsorption isotherm, and the pore size in nm indicated as “nm” is plotted along the abscissa for the mesopore size distribution displayed in the graph displaying the nitrogen adsorption isotherm. For the nitrogen adsorption, the values for the adsorption are indicated by the symbols “▪” whereas the values obtained for the desorption are indicated by the symbols “”, and the pore volume in ml/g is plotted in arbitrary units along the abscissa.

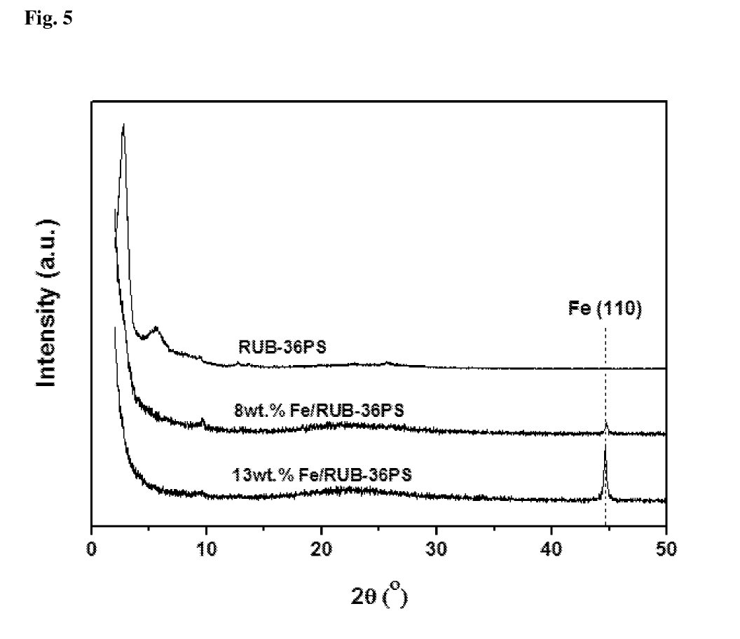

FIG. 5 displays the X-ray diffraction patterns of RUB-36 pillared silicate obtained according to Example 1 and the RUB-36 pillared silicate loaded with iron obtained according to Example 3 with a loading of 8 wt.-% and 13 wt.-% of iron, respectively. In the figure, the diffraction angle in ° 2Theta is designated as)“2θ(°)” and is plotted along the abscissa, and the diffraction intensity in arbitrary units is designated as “Intensity (a.u.)” and is plotted along the ordinate. In the Figure, the diffraction patterns of RUB-36 pillared silicate is designated as “RUB-36PS”, whereas the diffraction patterns of RUB-36 pillared silicate loaded with 8 wt.-% and 13 wt.-% of iron are designated as “8 wt. % Fe/RUB-36PS” and “13 wt. % Fe/RUB-36PS”, respectively. Furthermore, the (110)-reflection of elemental iron is indicated as a dashed vertical line and designated as “Fe (110)” for identification of the respective reflection resulting from elemental iron contained in the diffraction patterns of RUB-36 pillared silicate loaded with iron, respectively.

The invention is now described with reference to the following examples.

### EXAMPLES

### Example 1

### Preparation of an RUB-36 Interlayer Expanded Silicate

The layered silicate RUB-36 layered silicate was swollen using a cetyltrimethylammonium hydroxide (CTAOH) solution at room temperature (−27° C.). More specifically, 0.5 g RUB-36 was dispersed in the 35 g CTAOH solution (4%). The mixture was stirred for 48 h, then filtered and washed with deionized water, and finally dried at room temperature to obtain the RUB-36 interlayer expanded silicate.

### Preparation of a Pillared Silicate:

1.0 g of the RUB-36 interlayer expanded silicate was dehydrated under vacuum at 60° C. for 2 h, then protected under Ar, and subsequently refluxed in 10 ml tetraethylorthosilicate (TEOS) for 24 h at 78° C. The mixture was filtered and dried at room temperature, thus obtaining a white powder.

1.5 g of the interlayer expanded silicate treated with TEOS was hydrolyzed at 40° C. for 10 h in 20 ml of an aqueous solution of NaOH having a pH=8.0. The resulting mixture was filtered and washed with deionized water, and finally dried at room temperature.

The hydrolyzed sample was calcined under Ar (flow rate=30 ml/min) with a heating rate of 1° C./min and kept at 450° C. for 4 h. The atmosphere was then changed to air (flow rate=30 ml/min) and the sample was calcined at 550° C. for 5 h, thus affording the RUB-36 pillared silicate.

In FIG. 1 the X-ray diffraction pattern of the RUB-36 layered silicate used as starting material is displayed together with the RUB-36 interlayer expanded silicate and the RUB-36 pillared silicate respectively obtained according to the present procedure. As may be taken from the shift of the highest intensity (001) peak in the X-ray diffraction patterns displayed in FIG. 1, the interlayer expanded silicate is yet further expanded upon treatment and hydrolysis with TEOS for obtaining the pillared silicate.

In FIG. 2, the N₂ adsorption-desorption isotherm the RUB-36 pillared silicate is displayed, together with a plot of the mesopore size distribution as obtained by the BJH-method. The BET surface area of the RUB-36 pillared silicate was determined to 1,200 m²/g and the total pore volume to 0.73 cm³/g.

### Example 2

### Preparation of an RUB-36 Interlayer Expanded Silicate

The layered silicate RUB-36 was swollen by a mixture of an aqueous solution of cetyltrimethylammonium bromide (CTABr) and tetra-n-propylammonium hydroxide (TPAOH). More specifically, 0.5 g RUB-36 was dispersed in the solution containing 2.5 g CTABr, 1.0 g TPAOH and 44 g water. The mixture was stirred for 48 h at RT, then filtered and washed with deionized water, and finally dried at room temperature to obtain the RUB-36 interlayer expanded silicate.

### Preparation of an RUB-36 Pillared Silicate:

1.0 g of the RUB-36 interlayer expanded silicate was dehydrated under vacuum at 60° C. for 2 h, then protected under Ar, and subsequently refluxed in 10 ml tetraethylorthosilicate (TEOS) for 24 h at 78° C. The mixture was filtered and dried at room temperature, thus obtaining a white powder.

1.5 g of the interlayer expanded silicate treated with TEOS was hydrolyzed at 40° C. for 10 h in 20 ml of an aqueous solution of NaOH having a pH=8.0. The resulting mixture was filtered and washed with deionized water, and finally dried at room temperature.

The hydrolyzed sample was calcined under Ar (flow rate=30 ml/min) with a heating rate of 1° C./min and kept at 450° C. for 4 h. The atmosphere was then changed to air (flow rate=30 ml/min) and the sample was calcined at 550° C. for 5 h, thus affording the RUB-36 pillared silicate.

### Example 3

### Preparation of an RUB-36 Pillared Silicate Loaded with Iron

(NH₄)₄Fe(CN)₆ was employed as the iron precursor to prepare an RUB-36 pillared silicate loaded with iron. More specifically, RUB-36 pillared silicate loaded with 8 wt.-% iron was prepared by impregnating 0.6 g of dried RUB-36 pillared silicate obtained from Example 1 with 1.36 g of a 20% (NH₄)₄Fe(CN)₆ aqueous solution via incipient wetness, following by drying of the impregnated sample at room temperature, and then at 100° C. for 12 h. Finally the iron complex impregnated into the RUB-36 pillared silicate was decomposed under Ar at 630° C. for 4 h to afford RUB-36 pillared silicate loaded with 8 wt.-% iron.

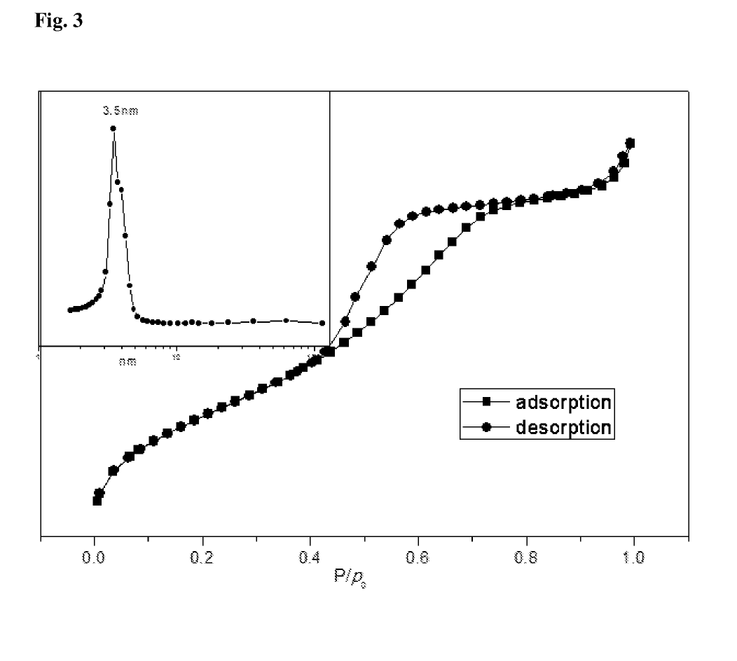

In FIG. 3, the N₂ adsorption-desorption isotherm the RUB-36 pillared silicate loaded with 8 wt.-% iron is displayed, together with a plot of the mesopore size distribution as obtained by the BJH-method. The BET surface area of the RUB-36 pillared silicate loaded with 8 wt.-% iron was determined to 365 m²/g and the total pore volume to 0.41 cm³/g.

In FIG. 5 the X-ray diffraction pattern of the RUB-36 pillared silicate used as starting material is displayed together with the RUB-36 pillared silicate loaded with 8 wt.-% iron obtained according to the present procedure. As may be taken from the reflection around 45° 2Theta in the X-ray diffraction pattern displayed in FIG. 5 for the sample obtained according to the present procedure, iron is present as elemental iron in the RUB-36 pillared silicate structure.

### Example 4

For obtaining RUB-36 pillared silicate loaded with 13 wt.-% iron, the RUB-36 pillared silicate loaded with 8 wt.-% iron obtained from Example 3 was subject to a further impregnation step wherein 0.22 g of the dried RUB-36 pillared silicate loaded with 8 wt.-% iron was impregnated with 0.29 g of a 20% (NH₄)₄Fe(CN)₆ aqueous solution via incipient wetness. The impregnated sample was then decomposed under Ar at 630° C. for 4 h to afford RUB-36 pillared silicate loaded with 13 wt.-% iron.

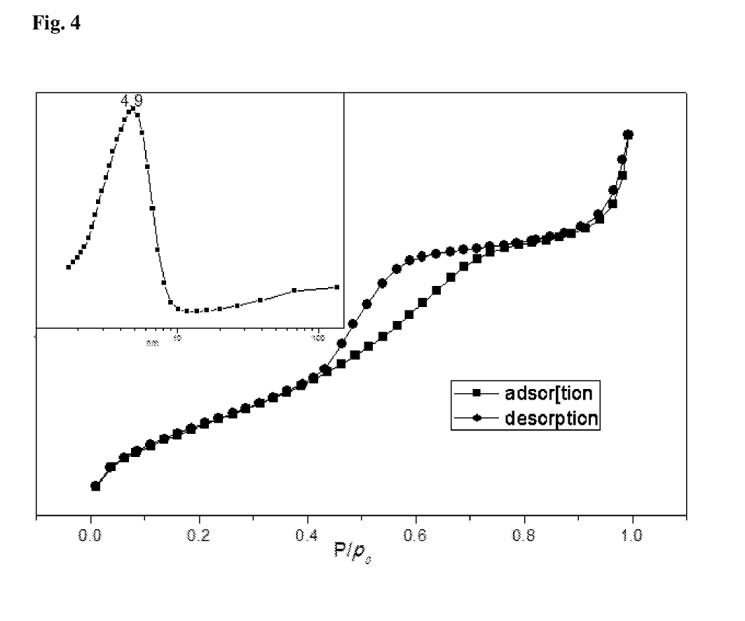

In FIG. 4, the N₂ adsorption-desorption isotherm the RUB-36 pillared silicate loaded with 8 wt.-% iron is displayed, together with a plot of the mesopore size distribution as obtained by the BJH-method. The BET surface area of the RUB-36 pillared silicate loaded with 13 wt.-% iron was determined to 303 m²/g and the total pore volume to 0.42 cm³/g.

In FIG. 5 the X-ray diffraction pattern of the RUB-36 pillared silicate loaded with 8 wt.-% iron used as starting material is displayed together with the RUB-36 pillared silicate loaded with 13 wt.-% iron obtained according to the present procedure. As may be taken from the X-ray diffraction pattern displayed in FIG. 5 for the sample obtained according to the present procedure, the Fe (110)-reflection around 45° 2Theta is accordingly more intense than in the starting material, indicating that iron from the further impregnation procedure is also present as elemental iron in the RUB-36 pillared silicate structure.

### Example 5

The procedure of Examples 3 was repeated, wherein after impregnation of the RUB-36 pillared silicate with (NH₄)₄Fe(CN)₆ aqueous solution via incipient wetness, followed by drying of the impregnated sample at room temperature and at 100° C. for 12 h, the sample was not calcined. Instead, the sample was treated with hydrogen gas at 400° C. for obtaining the RUB-36 pillared silicate structure loaded with 8 wt.-% of elemental iron.

### Example 6

The procedure of Examples 4 was repeated using the RUB-36 pillared silicate loaded with 8 wt.-% iron obtained from Example 5, wherein after impregnation with (NH₄)₄Fe(CN)₆ aqueous solution via incipient wetness, followed by drying of the impregnated sample at room temperature and then at 100° C. for 12 h, the sample was again not calcined. Instead, the sample was once more treated with hydrogen gas at 400° C. for obtaining the RUB-36 pillared silicate structure loaded with 12 wt.-% of elemental iron.

### Example 7

### Catalytic Testing of RUB-36 Pillared Silicate Loaded with Iron

Respective samples of iron supported catalyst obtained from Examples 3, 4, 5, and 6 were pelleted to 40-60 mesh at 8 MPa, and then loaded into a fix-bed catalytic evaluation device. Prior to testing, the catalyst was activated under H₂ (flow rate=30 ml/min) at 400° C. for 12 h. After the bed temperature had cooled down to about 270° C., syngas (CO:H₂=1) was fed to the evaluation device. The catalytic reaction was conducted under 0.5 Mpa of syngas (CO:H₂=1) with a gas space hour velocity (GSHV) of 1×10⁴ h⁻¹, and the temperature was elevated to 300° C. to begin the active test.

After passing the reactor, all gas lines were kept at 120° C. The effluents were analyzed using an online gas chromatograph (Agilent 7890A), which was equipped with a thermal conductivity detector (TCD) and a flame ionization detector (FID). Furthermore, four chromatography columns were installed (Porapak Q and 5 Å molecular sieves packed columns, and modified Al₂O₃ and FFAP capillary columns)

The results from catalytic testing are shown in Tables 1 and 2 below:

TABLE 1
Catalytic Results using Samples from Examples 3 and 4

| | Example 3 | Example 4 |
|---|---|---|
| wt.-% Fe | 8 wt.-% | 13 wt.-% |
| CO conversion (%) | 1.0 | 3.9 |
| CO₂ selectivity (%) | 7.6 | 14.9 |
| CH selectivity (%) | 86.7 | 78.8 |
| CH₄ | 26.9 | 19.5 |
| C₂⁼-C₄⁼ | 33.0 | 37.6 |
| (C₂⁼-C₄⁼)/( C₂⁰-C₄⁰) | 2.5 | 3.5 |
| C₅⁺ | 26.6 | 32.1 |

TABLE 2
Catalytic Results using Samples from Examples 5 and 6

| | Example 5 | Example 6 |
|---|---|---|
| wt.-% Fe | 8 wt.-% | 12 wt.-% |
| CO conversion (%) | 6.0 | 11.9 |
| CO₂ selectivity (%) | 16.2 | 25.1 |
| CH selectivity (%) | 81.2 | 72.8 |
| CH₄ | 21.1 | 16.4 |
| C₂⁼-C₄⁼ | 35.7 | 38 |
| (C₂⁼-C₄⁼)/( C₂⁰-C₄⁰) | 3.5 | 3.8 |
| C₅⁺ | 33.1 | 35.7 |

Thus, as may be taken from the results displayed in Tables 1 and 2, a highly selective catalyst for the conversion of syngas to olefins is provided by the present invention. Furthermore, as may be taken from the results obtained for Examples 3 and 4, the catalyst acitivity may be increased by a factor of almost 4 upon increasing the loading with iron from 8 to 13 wt.-%. Yet a further increase in the catalyst activity is observed for Examples 5 and 6, wherein the preparation of the catalyst contiaining elemental iron by reaction with H₂ at 400° C. (see Examples 5 and 6) as opposed to the preparation by calcination (see Examples 3 and 4). As regards said catalyst samples, an increase in activity by a factor of almost 2 is achieved by increasing the loading with elemental iron from 8 to 12 wt.-%. In all cases, an increase in the catalyst selectivity relative to the olefin product is observed when increasing the loading of the catalyst with elemental iron.

All references, including publications, patent applications, and patents, cited herein are hereby incorporated by reference for all purposes to the same extent as if each reference were individually and specifically indicated to be incorporated by reference and were set forth in its entirety herein.

The use of the terms “a” and “an” and “the” and similar referents in the context of describing the materials and methods discussed herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims.

What is claimed is:

1. A process for the production of a pillared silicate comprising:
(i) providing a layered silicate;
(ii) interlayer expanding the layered silicate provided in step (i) comprising a step of treating the layered silicate with one or more swelling agents;
(iii) treating the interlayer expanded silicate obtained in step (ii) with one or more hydrolyzable silicon containing compounds;
(iv) treating the interlayer expanded compound obtained in step (iii) with an aqueous solution to obtain a pillared silicate;
(v) removing at least a portion of the one or more swelling agents from the pillared silicate obtained in step (iv); and
(vi) impregnating the pillared silicate obtained in step (v) with one or more elements selected from the group consisting of Fe, Ru, Ir, and combinations of two or more thereof.

2. The process of claim 1, further comprising:
(vii) drying the impregnated pillared silicate; and/or
(viii) calcining the impregnated and optionally dried pillared silicate.

3. The process of claim 2, further comprising:
(ix) treating the impregnated and optionally dried and/or optionally calcined pillared silicate with a reducing agent.

4. The process of claim 1, wherein the interlayer expanded silicate obtained in step (ii) is dehydrated prior to step (iii).

5. The process of claim 1, wherein the layered silicate provided in step (i) comprises one or more layered silicate compounds selected from the group consisting of MCM-22, PREFER, Nu-6(2), CDS-1, PLS-1, MCM-47, ERS-12, MCM-65, RUB-15, RUB-18, RUB-20, RUB-36, RUB-38, RUB-39, RUB-40, RUB-42, RUB-51, BLS-1, BLS-3, ZSM-52, ZSM-55, kanemite, makatite, magadiite, kenyaite, revdite, montmorillonite, and combinations of two or more thereof.

6. The process of claim 1, wherein the layered silicate provided in step (i) is isomorphously substituted.

7. The process of claim 1, wherein the one or more swelling agents used in step (ii) comprise one or more compounds selected from the group consisting of cationic surfactants.

8. The process of claim 7, wherein the cationic surfactant contains 1 to 4 alkyl chains having 4 C-atoms or more.

9. The process of claim 8, wherein the 1 to 4 alkyl chains having 4 C-atoms or more include one or more C₄-C₂₆ alkyl chains.

10. The process of claim 7, wherein the cationic surfactant comprises one or more tetraalkylammonium compounds.

11. The process of claim 1, wherein the hydrolyzable silicon containing compound comprises one or more silicon compounds X¹X²SiX³X⁴, wherein X¹, X², X³, and X⁴ independently from one another represent a leaving group, wherein the leaving group X¹, X², X³, and X⁴ may be the same or different from one another.

12. The process of claim 1, wherein in step (iii) the treatment is conducted at a temperature in the range of from 25° C. to the refluxing temperature of the hydrolizable silicon containing compound in the mixture provided in step (iii).

13. The process of claim 1, wherein in step (iii) the treatment is conducted for a period of from 1 to 72 h.

14. The process of claim 1, wherein the aqueous solution used in step (iv) has a pH of from 5 to 10.

15. The process of claim 1, wherein step (v) includes a calcination step for removing at least a portion of the swelling agent.

16. The process of claim 2, wherein the calcination in step (viii) is conducted at a temperature ranging from 400 to 950° C.

17. The process of claim 1, wherein the one or more sources for the one or more elements in step (vi) comprise one or more metal compounds selected from the group consisting of metal salts, metal complexes, organometallic compounds, and combinations of two or more thereof.

18. The process of claim 17, wherein the metal complexes comprise a ligand selected from the group consisting of mono-, bi-, tri-, tetra-, penta-, and hexadentate ligands, including combinations of two or more thereof.

19. The process of claim 1, wherein step (vi) is repeated one or more times.

20. The process of claim 3, wherein the reducing agent in step (ix) comprises one or more compounds selected from the group consisting of hydrogen, hydrides, metals, sulfites, phosphites, hypophosphites, hydrazine, and combinations of two or more thereof.

21. The process of claim 1, wherein in step (vi) the element is Fe.

22. A pillared silicate obtained according to the process of claim 1.

23. A pillared silicate comprising an interlayer expanded layered silicate structure, wherein the BET surface area of the pillared silicate as determined according to DIN 66131 ranges from 900 to 1,500 m²/g.

24. The pillared silicate of claim 22, wherein the BET surface area of the pillared silicate as determined according to DIN 66131 ranges from 950 to 1,450 m²/g.

25. The pillared silicate of claim 22, further comprising one or more elements supported on the pillared silicate, wherein the one or more elements are selected from the group consisting of Fe, Ru, Ir, and combinations of two or more thereof.

26. The pillared silicate of claim 25, wherein the one or more elements are present in an amount ranging from 0.1 to 50 wt.-% based on 100 wt-% of the pillared silicate.

27. The pillared silicate of claim 22, wherein the layered silicate structure comprises silicate layers selected from the group consisting of zeolite-type layers.

28. The pillared silicate of claim 22, wherein the layered silicate structure originates from one or more layered silicate compounds and/or is derived or derivable from one or more layered silicate compounds.

29. The pillared silicate of claim 22, wherein the pillared silicate is isomorphously substituted.

30. A process for the production of one or more olefins comprising the steps of:
(1) providing a pillared silicate according to claim 22;
(2) contacting the pillared silicate with a gas stream comprising CO and H₂.

31. The process of claim 30, wherein the contacting in step (2) is performed at a temperature ranging from 100 to 700° C.

32. The process of claim 30, wherein the contacting in step (2) is performed at a gas hourly space velocity of the gas stream ranging from 0.01×10⁴ to 50×10⁴ h⁻¹.

33. The process of claim 30, wherein the CO:H₂ molar ratio in the gas stream ranges from 0.05:1 to 1:0.05.

34. The process of claim 30, wherein the contacting in step (2) is performed at a pressure ranging from 0.01 to 20 MPa.

35. A method of absorbing or ion-exchanging, the method comprising adding the pillared silicate of claim 22 as a molecular sieve, catalyst, catalyst component, catalyst support or binder thereof.
